(19) United States
(12) Patent Application Publication
(10) Pub. No.: US 2013/0011441 A1
(43) Pub. Date: Jan. 10, 2013

(54) TARGETED DELIVERY OF siRNA

(75) Inventors: Jeffrey O. Hollinger, Gibsonia, PA (US); Krzysztof Matyjaszewski, Pittsburgh, PA (US); Abiraman Srinivasan, Monroeville, PA (US)

(73) Assignee: Carnegie Mellon University, Pittsburgh, PA (US)

(21) Appl. No.: 13/345,494

(22) Filed: Jan. 6, 2012

Related U.S. Application Data
(60) Provisional application No. 61/460,703, filed on Jan. 6, 2011.

(51) Int. Cl. A61K 31/713; A61P 19/00; A61K 9/00; B82Y 5/00

(52) U.S. Cl. 424/400; 514/44 A; 977/795; 977/906

(57) ABSTRACT

The present invention relates to nanostructured bioconjugates and nano-structured network hydrogels used to deliver nucleic acids to targeted biological locations. The present invention further relates to methods of treating clinical conditions using the nanostructured bioconjugates and nano-structured network hydrogels.

### CROSS REFERENCE TO RELATED APPLICATIONS

The application claims the benefit of priority to U.S. Provisional Patent Application 61/460,703, filed on Jan. 6, 2011, the disclosure of the entirety of which is incorporated herein by reference.

### STATEMENT REGARDING FEDERALLY FUNDED RESEARCH

The invention was made with partial Government support under contract No. W81XWH-09-DMRDP-ARATDA from the Department of the Army. The government has certain rights in the invention.

### REFERENCE TO SEQUENCE LISTING

The application contains a Sequence Listing in accordance with 37 C.F.R. §§1.821-1.825. The material in the Sequence Listing text file is herein incorporated by reference in its entirety in accordance with 37 C.F.R. §1.52(e)(5). The electronically submitted Sequence Listing, entitled “110618 Sequence Listing_ST25.txt” contains one 2 Kb text file and was created on Jan. 5, 2012 using an IBM-PC machine format.

### TECHNICAL FIELD OF THE INVENTION

The present invention relates to nanostructured bioconjugates and nano-structured network hydrogels and methods of treating clinical conditions using the nanostructured bioconjugates and nano-structured network hydrogels.

### BACKGROUND

Inhibiting a target protein using selected siRNA's can effectively deregulate either the function of an individual gene or group of genes without eliciting a toxic or an immune response (i.e., an ‘off-target’ effect). Post-transcriptional gene silencing occurs through RNA interference, where the double stranded RNAs (dsRNAs) are cleaved into 21-23 nucleotide fragments (i.e., short interfering RNA: siRNA). Cleavage occurs by a cellular endonuclease of the ribonuclease-III type called DICER. The short duplexed siRNA's are unwound by a helicase with the antisense strand becoming incorporated into the multi-component RNA-induced silencing complex (RISC). This moiety mediates sequence-specific gene silencing by cleaving the target mRNA. The mRNA targets are the key nuclear transcriptional factors Runt related protein 2 (Runx2/Cbfa1) and Osterix (Osx) that cue osteoblast lineage progression. Consequently, silencing selective BMP-signaling genes using siRNA's provides a powerful therapeutic effect by preventing Heterotopic ossification (HO).

Heterotopic ossification (HO) is a compelling clinical concern for civilian and military medical practices. An estimated 150,000 total hip arthroplasties (THAs), knee surgeries, spine cage implantations and fusions, and shoulder procedures are performed each year and the incidence of HO may be as high as 90 percent [Iorio, et. al. J Am Acad Orthop Surg 2002, 10, (6), 409-16]. 20-30% of patients with neurologic deficits develop HO with as many as 50% of those patients with spinal cord injury developing HO.

The current therapeutic strategies to mitigate HO are rehabilitation [Vanden Bossche et. al, J Rehabil Med 2005, 37, (3), 129-36], surgery [van Ooij, et. al, Ned Tijdschr Geneeskd 2005, 149, (1), 37-41], radiation [Parkinson, et. al. Hip 1982, 211-27], bisphosphonates and non steroidal anti-inflammatory drugs (NSAIDs) [Vanden Bossche et. al, J Rehabil Med 2005, 37, (3), 129-36]. The morbidity associated with these therapies may lead to secondary complications [Vanden Bosscheet. al, J Rehabil Med 2005, 37, (3), 129-36]. Currently, there is no known therapeutic strategy to prevent HO. Therefore, wounded service members have a limited number of unsatisfactory therapeutic options once HO occurs at the amputation stump site. Treatments include surgical intervention to excise the HO, which often lead to additional HO and localized radiation, which is intrinsically deleterious.

Presently there is a robust and diverse library of candidate siRNA's to prevent HO and this required establishment of a screening paradigm of in vitro assays to determine the ‘best performing’ siRNA's measured against a standard anti-BMP: noggin. Traditional cell and molecular biology techniques were applied to bone studies to verify effectiveness of the siRNA suite and the leading siRNA candidates moved forward to the in vivo phase.

The siRNA phosphodiester backbone is an anionically charged molecule [Ma, et. al., Nature 2004, 429, (6989), 318-322.] and naked siRNA does not pass through the cell membrane [Akhtar, et. al., Adv. Drug Delivery Rev. 2007, 59, (2-3), 164-182]. The electrostatic repulsion between naked siRNA and the anionic cell membrane surface prevents naked siRNA endocytosis [Akhtar, et. al., Adv. Drug Delivery Rev. 2007, 59, (2-3), 164-182]. Therefore, a selective delivery system is required for efficient transportation of siRNA and release of the siRNA within the targeted cell. The most commonly used gene delivery systems (FIG. 4) can be divided into biological (viral) and nonbiological (non-viral) systems.

Biological carriers and viruses possess and provide efficiency in siRNA transfer but are difficult to produce and are toxic [Thomas, et; al., Nat. Rev. Genet. 2003, 4, (5), 346-358]. These limitations mean that development of non-biological systems for siRNA delivery remains a high priority. Non-viral delivery systems include peptides, lipids (liposomes), dendrimers and linear or branched polymers with cationic charges [Duncan, et al., Adv. Polym. Sci. 2006, 192, (Polymer Therapeutics I), 1-8] that interact with the negatively charged siRNA through electrostatic interactions [El-Aneed, J. Controlled Release 2004, 94, (1), 1-14]. Among non-viral delivery systems, dendrimers have the advantage of possessing well-defined structure, size, stability and biocompatibility [Duncan, et al., Adv. Drug Delivery Rev. 2005, 57, (15), 2215-2237]. However, the multistep synthesis and laborious purification at each step of the synthesis, and, consequently, high preparation cost of dendrimers limit their application. Prior art methods to synthesize biomedical polymers rely on a step-growth condensation polymerization protocol that may yield ill-defined polymers with high polydispersity, uncontrolled functionality, topology and composition, which are not ideal for siRNA delivery.

Therefore, there is a need for a nucleic acid delivery system that is readily produced and that can be used to efficiently deliver nucleic acids, such as siRNA, to a targeted biological location to treat various clinical conditions.

### BRIEF SUMMARY

One embodiment according to the present disclosure is directed to a nanostructured bioconjugate. The nanostructured bioconjugate comprises a polymeric nanostructure formed using a controlled radical polymerization process and a nucleic acid at least partially encapsulated by a cationic region of the polymeric nanostructure. The polymeric nanostructure comprises a cationic region, at least one degradable unit, and at least one moiety. The degradable unit is formed by the incorporation of a divinyl monomeric unit, wherein the vinyl units are connected directly or indirectly by a degradable linking group. The moiety is selected from the group consisting of a covalently incorporated tertiary amine moiety, a covalently incorporated quaternary ammonium moiety, and combinations of any thereof.

An additional embodiment according to the present disclosure is directed to a method of treating a clinical condition comprising delivering a nucleic acid to a targeted biological location using a nanostructured bioconjugate.

A further embodiment according to the present disclosure is directed to a nano-structured network hydrogel. The nano-structured network hydrogel comprises a nanostructured bioconjugate and a matrix forming compound, wherein the nanostructured bioconjugate and the matrix forming compound form a porous three-dimensional nano-structured network hydrogel in-vivo.

An additional embodiment according to the present disclosure is directed to a method of treating a clinical condition comprising: administering a nanostructured bioconjugate to a patient having a clinical condition, administering a matrix forming compound to the patient, and forming a nano-structured network hydrogel, wherein the nano-structured network hydrogel and the nanostructured bioconjugate degrade releasing the nucleic acid to a localized targeted biological site.

It is understood that the invention disclosed and described herein is not limited to the embodiments disclosed in this Summary.

### BRIEF DESCRIPTION OF THE DRAWINGS

The foregoing summary, as well as the following detailed description, will be better understood when read in conjunction with the appended figures. In the figures:

FIG. 1 illustrates a radiographic film image of heterotopic ossification (within dotted lines) in muscle proximal to an amputation stump.

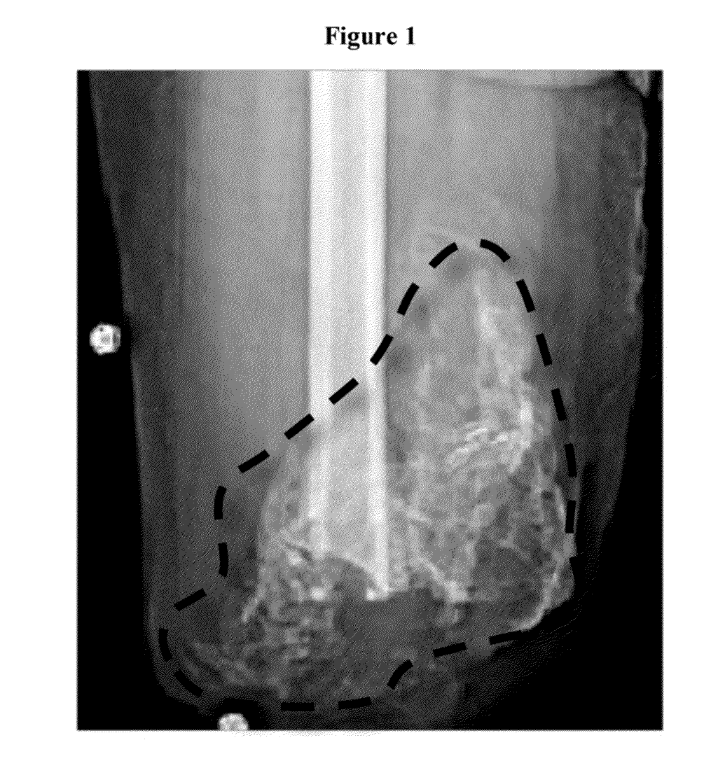

FIG. 2 illustrates exemplification of the present system for delivery of siRNA to inhibit osteoblast differentiation using an injectable nano-structured network hydrogel.

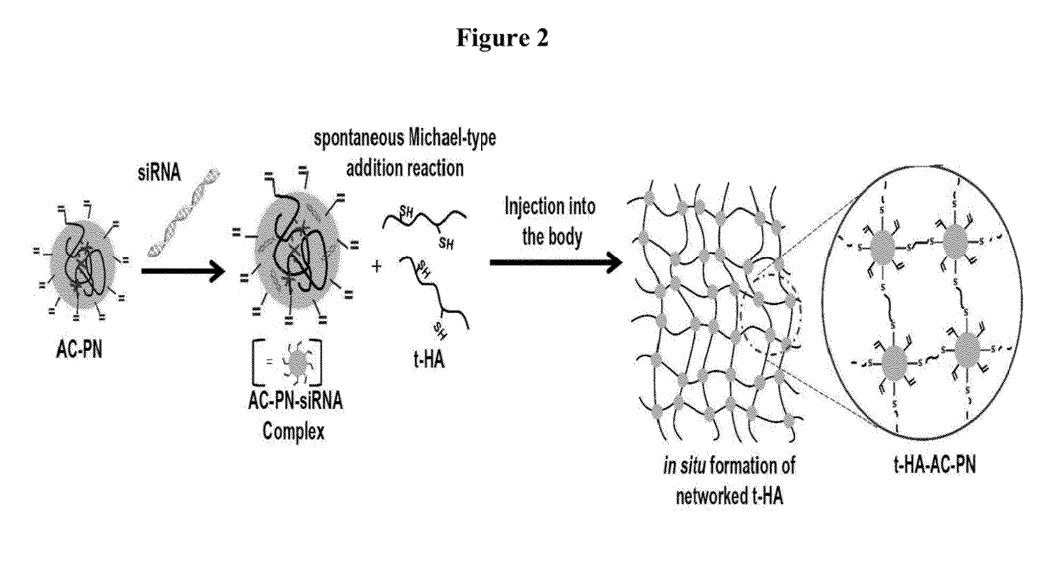

FIG. 3 illustrates a procedure for delivery of siRNA to targeted cells using an injectable nano-structured network hydrogel to inhibit osteoblast differentiation.

FIG. 4 illustrates statistics of prior art methods used for siRNA delivery.

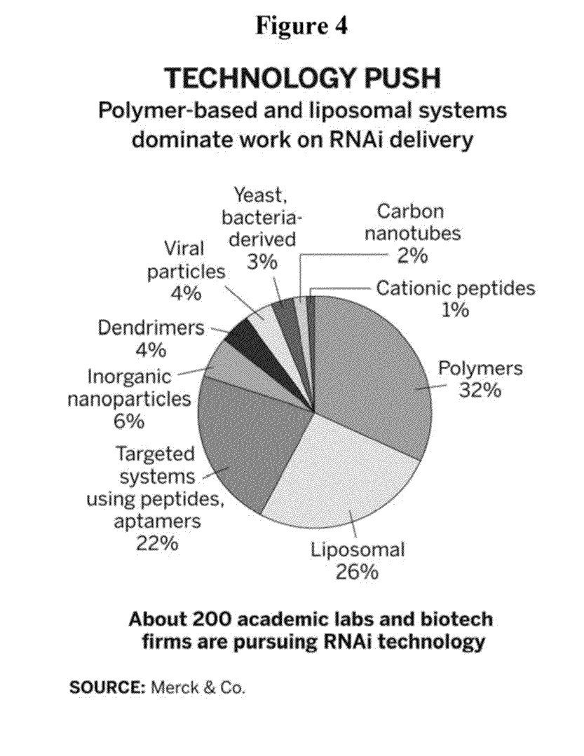

FIG. 5 illustrates a specific anti-siRNA delivery and confirmation of HO prevention strategy according to the present disclosure.

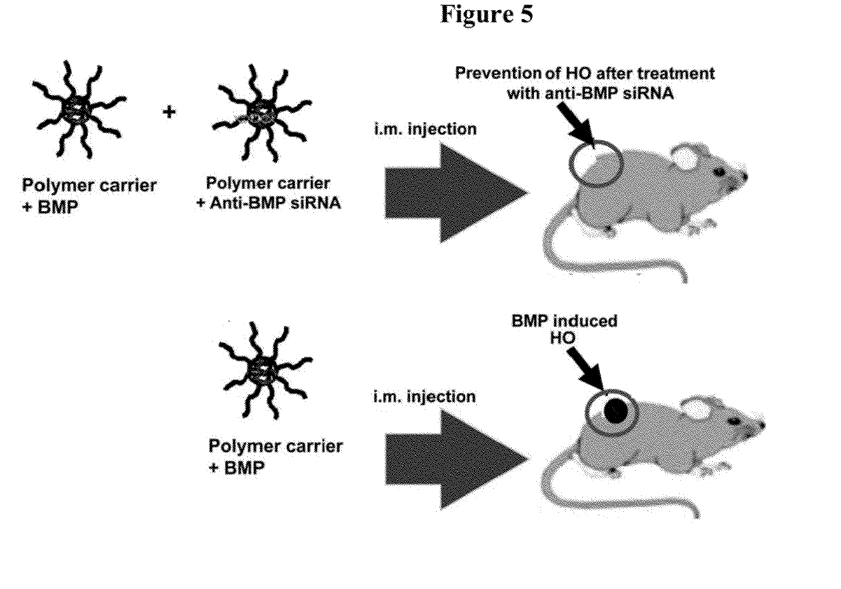

FIG. 6A illustrates a nano-gel structure.

FIG. 6B illustrates a star copolymer.

FIG. 6C illustrates a core-shell nano-gel.

FIG. 7 illustrates synthesis of hydrolysable acrylated rhodamine isothiocyanate-dextran (RITC-Dx) loaded POEO300MA-co-PHEMA nanogels by AGET ATRP in mini-emulsion polymerization.

FIG. 8 illustrates a fluorescence microscopy of MC3T3.E1.4 cells after 6 hours of incubation with bovine serum albumin (BSA)-loaded nanogels presented BSA protein identified by the fluorescent isothiocyante (FITC)-conjugated antibody for BSA and nuclei stained with Hoechst stain, suggesting internalization of the BSA loaded nanogel. The yellow arrow refers to the BSA protein and the blue arrow refers to the nuclei.

FIG. 9 illustrates a spheroid co-culture model. Merged differential interference contrast (DIC) and fluorescent confocal images of a control (a) spheroid cultured with FITC-Dx-loaded nanogels for 1 h (b). Confocal images of spheroids cultured in the presence of FITC-Dx-loaded nanogels without GRGDS (c) and with GRGDS (d) after 2 hrs. The optical section shown was taken at 28 μm cell depth into the co-culture spheroid that shows uniform internalization of nanogels in all the cells. An arrow points to a nanogel inside a cell.

FIG. 10 illustrates scanning electron microscope (SEM) micrographs of MC3T3-E1.4 pre-osteoblast cells attachment on GRGDS-(PEO)n-polyEGDMA functionalized nano-structured star polymer-coated surfaces.

FIG. 11 illustrates results of cell internalization of fluorescent PEO-poly(EGDMA) with MC3T3.E1.4 cells after 24 hours (A,D); Confocal microscopy suggests poor internalization efficiency of PEO nano-structured star polymers without GRGDS (A) when compared PEO stars with GRGDS (B and C). Flow cytometry suggested 100% internalization of PEO nano-structured star polymers with GRGDS compared to nano-structured star polymers without GRGDS.

FIG. 12 illustrates the formation of structured gel via Michael-type addition reactions under physiological conditions, the nano-structured hybrid hydrogel was visually observed with digital images before and after gelation.

FIG. 13 illustrates SEM photomicrographs of nano-structured HA hydrogel: (a and b) cross-section of interior, and surface hybridized with nanogels at different magnifications. The arrow points to a nanogel in HA scaffold.

FIG. 14 illustrates zeta potential analysis of nanostructured bioconjugates, which demonstrates a value shift from cationic (left peak) to anionic charge after siRNA complexation (right peak).

FIG. 15 illustrates a cellular internalization of nanostructured bioconjugates after 48 hrs obtained by confocal microscopy confirming robust internalization of FITC conjugated siRNA polyplex into C2C12 cells (A), the control group incubated with nano-structured polymer without FITC conjugated siRNA did not show any fluorescence (B).

FIG. 16 illustrates the synthesis of short-arm PEG (DP=22) stars with cationic core.

FIG. 17 illustrates a proposed AC-PN core-shell nanogel designed for complexation with siRNA.

FIG. 18A illustrates the synthesis of core-shell AC-PN by a miniemulsion with P(OEOMA₄₇₅)₄₅-b-P(MEMA)₁₀₆ reactive surfactant.

FIG. 18B illustrates cationic nanogel synthesis via AGET ATRP in inverse miniemulsion.

The patent or application file contains at least one drawing executed in color. Copies of this patent or patent application publication with color drawings will be provided by the office upon request and payment of the necessary fee.

### DETAILED DESCRIPTION

It is to be understood that certain descriptions of the present disclosure have been simplified to illustrate only those elements and limitations that are relevant to a clear understanding of the present disclosure, while eliminating, for purposes of clarity, other elements. Those of ordinary skill in the art, upon considering the present description, will recognize that other elements and/or limitations may be desirable in order to implement embodiments of the present disclosure. However, because such other elements and/or limitations may be readily ascertained by one of ordinary skill upon considering the present description, and are not necessary for a complete understanding of the present invention, a discussion of such elements and limitations is not provided herein. As such, it is to be understood that the description set forth herein is merely exemplary to embodiments of the present description and is not intended to limit the scope of the of the invention as defined by the claims.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about”, even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (end points may be used).

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

All referenced patents, patent applications, publications, sequence listings, electronic copies of sequence listings, or other disclosure material identified herein are incorporated by reference in whole but only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Certain embodiments according to the present disclosure are directed to a nanostructured bioconjugate. As used herein, the terms “nanostructured bioconjugate”, “cationic nanostructured bioconjugate”, “polyplex”, and “nano-structured polyplex” may be used interchangeably throughout the present application and claims. The nanostructured bioconjugate comprises a polymeric nanostructure formed using a controlled radical polymerization process and a nucleic acid at least partially encapsulated by a cationic region of the polymeric nanostructure. In certain specific embodiments, the controlled radical polymerization process is an atom transfer radical polymerization process (ATRP).

In certain embodiments, the polymeric nanostructure comprises a cationic region, at least one degradable unit, and at least one moiety. As used herein, the terms “polymeric nanostructure” and “nano-structured polymer” may be used interchangeably throughout the present application and claims. Examples of nano-structured polymers suited for use in the present disclosure include those described in U.S. application Ser. No. 12/311,673, the disclosure of which is incorporated in its entirety by this reference.

Degradable linking groups, such as biodegradable or cleavable crosslinkers, include peptides, [Khelfallah, N. S.; Decher, G.; Mesini, P. J. Macromolecular Rapid Communications 2006, 27, 1004-1008] anhydrides, [U.S. application Ser. No. 10/034,908] and oligo(lactate) esters [Huang, X.; Lowe, T. L. Biomacromolecules 2005, 6, 2131-2139]. These crosslink agents and the resulting hydrogels are degraded to water-soluble polymers. Disulfides of the type R—S₂—R (both linear or cyclic) present another class of (bio)degradable groups which can be cleaved to the corresponding thiols in the presence of reducing agents, such as, but not limited to, tributyl phosphine (Bu₃P), tris(2-carboxyethyl)phosphine (TCEP), dithiothreitol (DTT) and biological reducing agents present in the body. [Houk, J.; Whitesides, G. M. J. Am. Chem. Soc. 1987, 109, 6825; Tsarevsky, N. PhD Thesis CMU 2005, Chapter 6]. Other degradable links such as hydrazides, hydrazines, hydrazones, acetals, oximes, imines, Schiff bases or urethanes, while not as biologically benign may also be used to target different rates of degradation in different environments, as can crosslinking agents comprising degradable oligo/polymer segments such as a polysaccharide, polyesters, a peptide or protein, chitin, or chitosan. In certain other embodiments, the at least one degradable unit may be formed by the incorporation of at least one divinyl monomeric unit into the polymeric structure, wherein the vinyl units of the divinyl monomer unit are connected directly or indirectly by a degradable linking group. As used herein, the term “directly” refers to a vinyl unit covalently bonded directly to an atom of the degrading unit. As used herein, the term “indirectly” refers to a vinyl unit linked to an atom of the degrading unit by a chain comprising one or more covalently bonded atoms. In certain other embodiments, the degradable linking group is a degradable group selected from the group consisting of a disulfide group, an ester group, and acetal group, and combinations of any thereof. Since controlled radical polymerization processes are envisioned as one process for the preparation of the functional gels, a series of disulfide-functionalized dimethacrylate crosslinkers have been developed to exemplify the preparation of (bio)degradable bulk gels and gel particles. The degradation of disulfides has been utilized for the preparation of various polymeric materials, including stimulus-responsive gelators, [Li, C.; Madsen, J.; Armes, S. P.; Lewis, A. L. Angew. Chem. Int. Ed. 2006, 45, 3510] reversible shell-crosslinked micelles, [Li, Y.; Lokitz, B. S.; Armes, S. P.; McCormick, C. L. Macromolecules 2006, 39, 2726] miktoarm star copolymer, [Gao, H.; Tsarevsky, N. V.; Matyjaszewski, K. Macromolecules 2005, 38, 5995] and polymer capsules. [Zelikin, A. N.; Quinn, J. F.; Caruso, F. Biomacromolecules 2006, 7, 27]

The various embodiments of the nano-structured polymer comprise at least one moiety. According to certain embodiments, the at least one moiety may comprise a functional group capable of binding to the nucleic acid, for example by a cleavable covalent bond or a non-covalent interaction, such as, for example, an ionic bond or a electronic interaction. The moiety may act to form the cationic region within the polymeric nanostructure or a region within the polymeric nanostructure that can bond to or bind the nucleic acid within (i.e., at least partially encapsulate) the polymeric nanostructure. The at least one moiety may be installed in the polymer by copolymerizing using a monomer comprising the moiety when forming the polymeric nanostructure. In certain other embodiments, the moiety may be selected from the group consisting of a covalently incorporated tertiary amine moiety, a covalently incorporated quaternary ammonium moiety, and combinations of any thereof. For example, a moiety comprising a quaternary ammonium moiety may be installed using at least one radically copolymerizable monomer having a quaternary ammonium functional moiety. Alternatively, a moiety comprising a tertiary amine moiety may be installed using at least one radically copolymerizable monomer having a tertiary amine (which may be suitably protected, if necessary). In certain embodiments, the tertiary amine may be converted to a quaternary amine moiety subsequent to the formation of the nano-structured polymer, for example, by alkylation or protonation under acidic and/or physiological conditions.

A standard nomenclature was developed to avoid confusion among the many embodiments of suitable polymeric nanostructures synthesized to exemplify aspects of this disclosure. The first term in the nomenclature is NSP, which stands for Nano-Structured Polymer and this term applies to all of the polymeric nanostructures synthesized to examine the selective delivery concept discussed herein. The purpose of its inclusion is to indicate materials that are synthesized in-house for future comparison to commercially available materials. The second term describes the type of NSP which can be used in the various embodiments described herein, which includes polymeric nanostructures that can be described as star polymers (S), nanogels (NG) or core-shell nanogels (CS). The backbone of the polymeric nanostructure is included as a subscript of this term to indicate whether the polymer is either degradable or non-degradable. If degradable disulfide units are incorporated, the subscript is SSX and if non-degradable ethylene glycol dimethacrylate (EGDMA) units are incorporated, the subscript is EGDMA. The final term describes the charge of the NSP, where N describes a Neutral molecule, C a Cationic and qC for Quaternized Cationic moiety, e.g., when in the exemplary composition discussed herein incorporates a quaternized 2-(dimethylamino)ethyl methacrylate (DMAEMA) then qC is used instead of “C” when DMAEMA is incorporated.

This nomenclature is clarified in the following examples of the polymeric nanostructures prepared to exemplify the invention.

1) Star polymers with degradable disulfide backbone

a. Neutral Core: NSP-S_{SSX}-N

b. 5% DMAEMA Core: NSP-S_{SSX}-C

c. 5% quaternized DMAEMA Core: NSP-S_{SSX}-qC

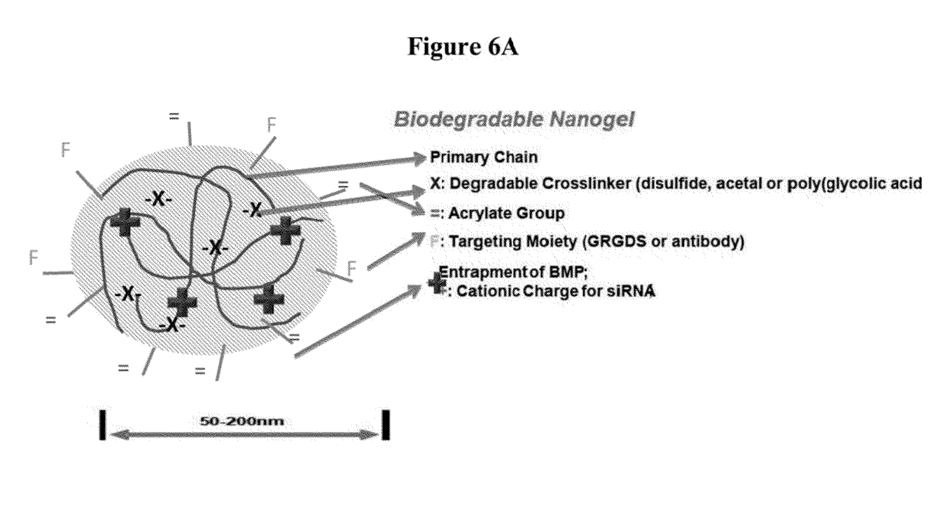

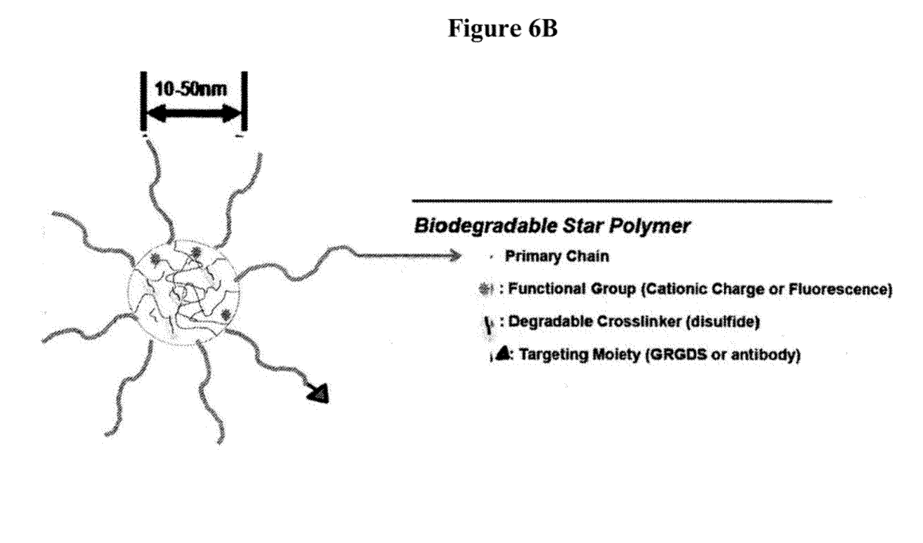

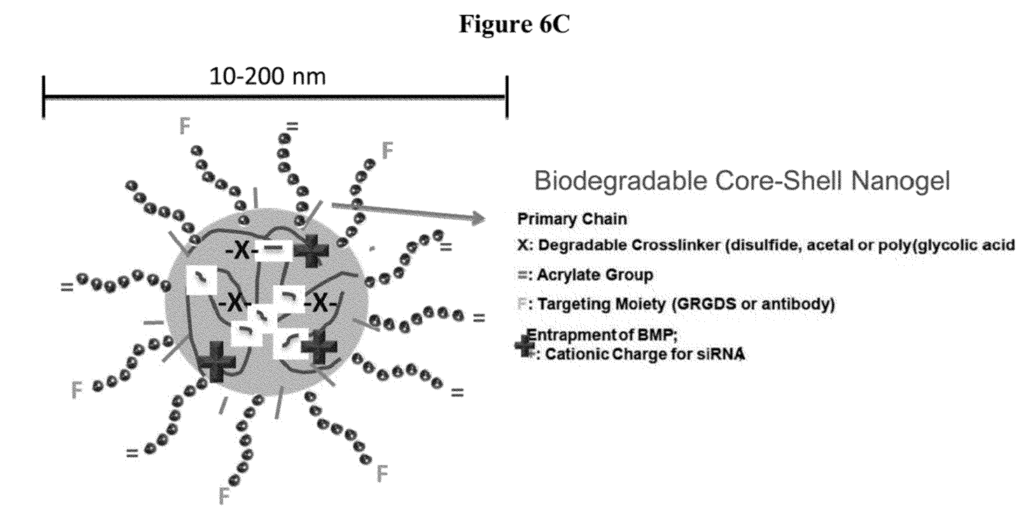

2) Star polymers with non-degradable backbone (See FIG. 6B)

a. 5% DMAEMA Core: NSP-S_{EGDMA}-C

### Characteristics:

1) Size: small (10-50 nm)

2) Loading capacity: lowest

3) Internalization: fastest

4) Entrapment: none

3) Nanogels with degradable disulfide backbone (See FIG. 6A)

b. Neutral Core: NSP-NG_{SSX}-N

c. 5% DMAEMA Core: NSP-NG_{SSX}-C

d. 5% quaternized DMAEMA Core: NSP-NG_{SSX}-qC

### Characteristics:

1) Size: large ˜100-300 nm

2) Loading capacity: highest

3) Internalization: slowest

4) Entrapment: highest

4) Core-shell nanogels with degradable disulfide backbone (See FIG. 6C)

e. Neutral Core: NSP-CS_{SSX}-N

f. 5% DMAEMA Core: NSP-CS_{SSX}-C

g. 5% quaternized DMAEMA Core: NSP-CS_{SSX}-qC

### Characteristics:

1) Size: medium ˜100-200 nm

2) Loading capacity: moderate

3) Internalization: moderate (hypothetically)

4) Entrapment: moderate

U.S. Published Application 2010/0143286 (Ser. No. 12/311,673), which is incorporated by reference in its entirety, further demonstrates various strategies related to the synthesis of certain embodiments of the polymeric nanostructures, for example, the nano-gel structure and the star copolymer. The present nano-structured polymers of the present disclosure may further include a cationic region or core, which may be utilized to bind to anionic nucleic acid functionality, such as a DNA or RNA structure. As used here, the terms “nano-gel structure”, “nanogel” “acrylated cationic nanogels”, “AC PN nanogels”, and “AC nanogels” may be used interchangeably throughout the present application and claims. As used here, the terms “star co-polymer”, “cationic star copolymer”, “nano-structured star polymer” “acrylated cationic star polymer (AC star)”, and “star polymer” may be used interchangeably throughout the present application and claims.

Delivery of nucleic acids, such as certain siRNA into complex tissue structures is a complicated task. In certain embodiments, a nanostructured bioconjugate structure described herein including the polymeric nano-structure may be used to protect the nucleic acid from enzymatic or chemical degradation and to enhance cellular uptake at the selected biological site. For example, the nanostructured bioconjugate structure may protect siRNA from RNase enzyme degradation and may be designed to enhance cellular uptake, for example, by having an exterior capable of crossing the cell wall. For example, in certain embodiments, a nanostructured bioconjugate comprises a polymeric nanostructure and a nucleic acid at least partially encapsulated by the cationic region of the polymeric nanostructure. As used herein, the term “nucleic acid” refers to a polymer of ribonucleic acids or deoxyribonucleic acids, including RNA, mRNA, rRNA, tRNA, small nuclear RNAs, short interfering ribonucleic acids (siRNAs), cDNA, DNA, PNA, RNA/DNA copolymers, or analogues thereof. A nucleic acid may be obtained from a cellular extract, genomic or extragenomic DNA, viral RNA or DNA, or artificially/chemically synthesized molecules. In certain embodiments, the nucleic acid comprises a short interfering ribonucleic acid (siRNA). In certain embodiments, the cation region of the polymeric nanostructure may interact with the siRNA by electrostatic interactions to bind the siRNA within the nanostructured polymer during formation and delivery of the nanostructured bioconjugate to the active site.

The siRNA may be chosen to inhibit certain RNA structures characteristic of specific clinical conditions. For example, in certain embodiments specific for treating heterotopic ossification, the siRNA may be selected to inhibit an RNA characteristic of HO, such as an RNA selected from the group consisting of a Runx2 mRNA, Osx mRNA, BMP type I receptor mRNA, BMP type II receptor mRNA, Transcriptional co-activator with PDZ binding motif (TAZ) mRNA, Promyelotic leukemia zinc finger (PLZF) mRNA, Mothers against decapentaplegic homologs (SMAD4) mRNA and combinations of any thereof. In certain specific embodiments, the siRNA may have a nucleic acid sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15 and combinations of any thereof. The present disclosure should be considered to include embodiments comprising analogous siRNA sequences which may contain one or more nucleotide change or substitution, such as a mutation, which does not significantly alter the activity or binding characteristics of the siRNA.

According to various embodiments, an siRNA therapeutic strategy directed selectively against Runx2 and/or Osx mRNA may not only prevents osteoblast differentiation induced by BMPs but may also prevent differentiation by other hormones, for example, the parathyroid hormone (PTH) and local growth factors, for example, fibroblast growth factor (FGF). This procedure exploits prophylactic administration and is therefore a preventative therapy for HO.

In one embodiment, noggin protein was selected as an anti-BMP biomolecule to compare the therapeutic efficiency to that of siRNA's in abrogating BMP-induced osteoblast differentiation. Under-expression of noggin augments ectopic ossification in Fibrodysplasia Ossificans Progressiva (FOP) patients. FOP is a mechanistic analog to HO. Therefore, therapeutic HO prevention is validated through the highly reproducible ectopic mouse muscle model and an FOP model.

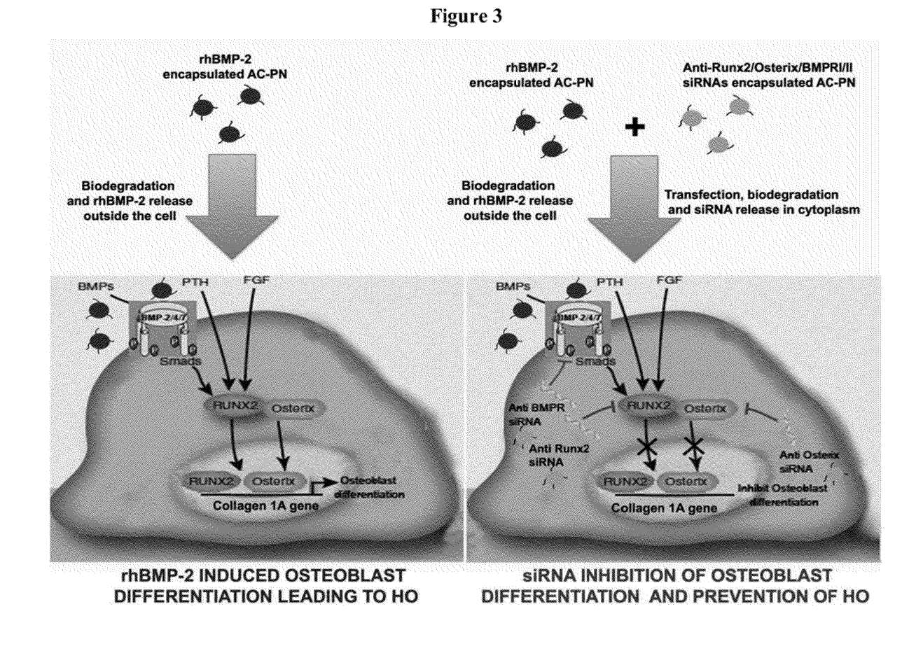

Osteoblast differentiation may be promoted by BMPs, but also by parathyroid (PTH) and growth hormones as well as local growth factors transforming growth factor-beta (TGF-β), insulin-like growth factor-I (IGF-I), platelet-derived growth factor (PDGF) and/or fibroblast growth factor (FGF) [Goldring, M. B.; Goldring, S. R., Clin Orthop Relat Res 1990, (258), 245-78]. The signaling pathways for these biomolecules converge at the nuclear transcriptional levels, as shown in FIG. 3. Therefore, in one example, osteoblast cell differentiation will be blocked by selectively knocking down runt-related protein 2 transcription factor (Runx2) [Colla, et. al., Leukemia 2005, 19, (12), 2166-76.] (upstream) and Osx [Ohyama, et. al., Endocrinology 2004, 145, (10), 4685-92] (downstream) with Anti-Runx2 and Anti-Osx siRNA's to stop the osteoblast differentiation signaling cascade and thus prevent HO. Runx2, also referred to as core binding factor alpha (Cbfa)1, participates in osteoblast differentiation. siRNA directed against Runx2 proteins will inhibit osteoblast differentiation and subsequent bone formation [Colla, et. al., Leukemia 2005, 19, (12), 2166-76]. Osterix is a transcription factor expressed during osteogenic cell differentiation and is osteoblast-specific and siRNA targeted against Osx will reduce osteoblast differentiation. BMP-2 and BMP-4 ligands bind to type-I receptors of BMP [Heldin, et al. Nature 1997, 390, (6659), 465-71].

In one example described below, site specific delivery of siRNA directed against BMP type I/II receptor proteins will stop BMP-2 induced osteoblast differentiation and thus inhibit osteogenesis. The concept of a suite of anti-BMP signaling molecules (i.e., siRNA-anti-Runx2, -anti-Osx and -anti-BMP type I/II receptors) is a key emphasis of this unique approach providing exemplification of a broadly applicable therapeutic design. Moreover, the emphasis and approach are consistent with bone induction biology. Consequently, in one embodiment, siRNA's may be custom-designed to target mRNAs from mesenchymal cells and osteoblast lineage cells that are typical for the healing bone wound and ectopic (i.e., heterotopic) ossification. A therapeutic strategy to prevent HO must focus on the cell lineage progression biology and signaling molecules at a targeted site. This non-limiting example is driven by the understanding and appreciation of the complex cellular and molecular biology of the HO process as well as the technical ability to direct the therapeutic locally and precisely to muscle cells at the site where it is needed to abrogate HO.

A diverse library of candidate siRNA's was tested to see which would most effectively prevent HO, Table 1. Consequently, the study design for this application established a screening paradigm of in vitro assays to determine the ‘best performing’ siRNA's measured against a standard anti-BMP: noggin. Traditional cell and molecular biology techniques that have been applied to bone studies that verify effectiveness of the siRNA suite were used. The purpose for the screening was to identify the leading siRNA candidate(s) that were subsequently evaluated in the in vivo phase. In one example, the three best performing siRNA candidates emphasize detection and measuring osteoblast markers Runx2, Osx, Osteocalcin (OCN) and alkaline phosphatase (ALP). Moreover, the process of building the optimal HO preventative therapy is determined systematically through standard in vitro assays that transitioned to the in vivo systems.

TABLE 1
Target genes and their associated pathways for microarray experiments.

| Target Gene | Associated Pathway |
|---|---|
| hBMP2 | Heterotopic Ossification |
| hGPX1 | Oxidative Stress and Antioxidant Defense |
| hGPX2 | Oxidative Stress and Antioxidant Defense |
| hGPX3 | Oxidative Stress and Antioxidant Defense |
| hSOD1 | Oxidative Stress and Antioxidant Defense |
| hSOD2 | Oxidative Stress and Antioxidant Defense |
| hSOD3 | Oxidative Stress and Antioxidant Defense |
| hAPOE | Oxidative Stress and Antioxidant Defense |
| hCat | Oxidative Stress and Antioxidant Defense |
| hTNF | Apoptosis |
| hTNFSF10 | Apoptosis |
| hCD70 | Apoptosis |
| hCASP1 | Apoptosis |
| hCASP2 | Apoptosis |
| hCASP3 | Apoptosis |
| hBAG1 | Apoptosis |
| hBAG3 | Apoptosis |
| hBCL2 | Apoptosis |

In certain embodiments, the nanostructured bioconjugate may further comprise a water soluble neutral polymeric layer comprising water soluble monomer units, for example a polymeric layer on the exterior surface of the nanostructured bioconjugate. In certain specific embodiments, the water soluble monomer units may be a monomer having a poly (alkylene oxide) structure, such as a poly(ethylene oxide). Controlled radical polymerization provides an excellent mechanism to control the polymer structure and install water soluble components at a portion of the polymer chain to form a layer in the formed polymeric nanostructure and consequently in the nanostructured bioconjugate. In certain embodiments, the nanostructured bioconjugate further comprises a water soluble neutral polymeric layer wherein a peripheral functionality of the water soluble neutral polymeric layer delivers a nucleic acid to a targeted biological location. As used herein, the term “water soluble neutral polymeric layer” refers to the peripheral region of the nanostructured bioconjugate that includes the moieties that are capable of allowing the nanostructured bioconjugate to cross cellular walls In certain embodiments, the nanostructured bioconjugate further comprises a water soluble neutral polymeric layer further comprising functionalized polymeric arms, wherein the functionalized polymeric arms comprise moieties used to target specific cells, such as an attached antibody or other targeting group.

Nano-structured star polymers and nanogels particles having a poly(ethylene oxide) (PEO) polymeric layer are biocompatible and can be peripherally functionalized to target cell specific receptors, which is a requirement in certain embodiments for selective siRNA delivery to target cells. In one exemplification of the process the injectable, biodegradable hydrogel scaffolds hybridized with the nanostructured bioconjugate disclosed herein offers the advantage of selective, in situ network formation resulting in formation of a degradable network that functions as a biocompatible matrix for cell and protein encapsulation and delivery of bioactive molecules, such as siRNA, to block BMP signaling. According to one embodiment, an acrylated cationic star polymers (AC star) or acrylated nanogel (AC-NG) or acrylated core-shell nanogel (AC-CS) can be designed to provide a robust affinity to the negatively charged siRNA's and will form a ‘polyplex’ with the agents and deliver specific moieties to abrogate BMP signaling, examples include anti-Runx2, Osx and BMP type-I/II siRNA's into target cells.

According to one embodiment, preparation of acrylated cationic nanogels by ATRP in inverse mini-emulsion provides control over particle size (between 50 and 200 nm in diameter), forming particles with uniform degradation kinetics, controlled release of encapsulated biomolecules and colloidal stability, FIG. 6A, [Oh, J. et al., Journal of the Amer. Chem. Soc., 2007, 129, (18), 5939-5945; Oh, J. et al., Journal of the Amer. Chem. Soc., 2006, 128, (16), 5578-5584]. In certain embodiments, the polymeric nanostructure may be a nano-gel structure having size in the range of about 25 nm to about 500 nm. The cationic nature of the nanogel, for example, at the cationic region, promotes complexation with negatively charged siRNA through electrostatic interactions for protection, stability and controlled release of siRNA. The free volume within the core and the number and distribution of the complex forming units within the core of the designed nanogel particle provides the ability to provide a matrix for controlled intramolecular complexation with the selected siRNA.

In another embodiment, an acrylated cationic star polymer (AC Star) may be prepared by an ATRP process with a well-defined size (between 10 and 50 nm in diameter) having a cationic degradable core for encapsulation and release of siRNA, FIG. 6B. In certain embodiments, the polymeric nanostructure may be a star copolymer having size in the range of about 10 to about 50 nm.

One benefit of an AC star compared to an AC nanogel, in certain embodiments, is the AC star has radiating functionalized arms [Gao, H. et al., Prog. Polym. Sci., 2009, 34, (4), 317-350; Oh. J. et al., Progress in Polymer Science, 2008, 33, (4), 448-477] of pre-determinable molecular weight which in certain embodiments can incorporate a peripheral targeting ligand or group that can be used for specific targeted cell-delivery, for example in certain embodiments to transfect the undifferentiated muscle stem cells contiguous to the targeted site via receptor mediated endocytosis. In certain specific embodiments, the polymeric nanostructure is a star copolymer where the star polymer comprises functionalized polymeric arms, wherein the polymeric arms comprise moieties used to target specific cells.

According to specific embodiments, the primary chains of the final stars or AC-PN nanogels or CS nanogels may be synthesized using ATRP. Biocompatible methoxyethoxyethoxy methacrylate (PMEO2MA) and oligo(ethylene oxide) methacrylate may be introduced into the functional copolymer precursors for the AC nanogel and methacrylated poly(ethylene oxide) chains (PEO-MA) for the AC star to produce a nanostructure bioconjugate having a water soluble neutral polymeric layer comprising water soluble monomer units. In specific embodiments, methacrylate cross linkers with specific degradable segments (exemplified by disulfide, acetal, and poly(glycolic acid)) may be incorporated into the AC-PN for controlled bio-environmental degradation and controlled release of biomolecules. For example, an acetal based cross linker may be selected that degrades either hydrolytically or in response to certain pH conditions. In various embodiments, the AC-PN can be designed to degrade into individual polymeric chains with a Mn=10,000 to 30,000 and a narrow molecular weight distribution, this sized moiety is within the renal excretion limit [Guimaraes, M., Biochim. Biophys. Acta, Gen. Subj., 1997, 1335, (1-2), 161-172]. In certain embodiments, the polymeric nanostructure may degrade to a degradation unit comprising a primary polymer chain length defined by the molar ratio of co-monomers to initiator and is in the range of Mn <30,000. In other embodiment, the polymeric nanostructure may degrade to a degradation unit that is below the renal threshold (i.e., the degradation unit may be readily removed by the kidney). The size of the AC-PN nanogel can be controlled by the amount and composition of surfactant used in the synthesis of the AC nanogel particles forming nano-material ranging between 10 and 200 nm facilitates transfection into cells, either through the transmembrane channels of the cell membrane or by endocytosis.

Certain embodiments refer to a method of treating a clinical condition comprising: delivering a nucleic acid to a targeted biological location using a nanostructured bioconjugate. In certain embodiments, the clinical condition is a pathological condition, an oncological condition, a genetic condition, and a vectoral condition. In certain specific embodiments, the clinical condition is heterotopic ossification. In certain other specific embodiments, the clinical condition is craniosynostosis. In certain other specific embodiments, the clinical condition is Fibrodysplasia Ossificans Progressiva (FOP).

In certain embodiments, the method of treating a clinical condition further comprises co-administering a matrix forming compound, wherein the nanostructured bioconjugate and the matrix forming compound form a porous three-dimensional nano-structured network hydrogel. In certain embodiments, the matrix forming compound is selected from the group consisting of thiolated hyaluronic acid, thiolated collagen, non-thiolated collagen and combinations of any thereof. In certain other embodiments, the nanostructured bioconjugate and the matrix forming compound react to form a porous three-dimensional nano-structured network hydrogel connected by a plurality of degradable cross-linking connections. For example, the nanostructure bioconjugate may be formulated to have functionality, such as Michael receptors or thiol functionality, on the periphery of the structure which can form disulfide bonds degradable cross-linking connections with the thiols in the thiolated hyaluronic acid and/or the thiolated collagen. In certain other specific embodiments, the nano-structured network hydrogel is capable of delayed delivery of the nucleic acid by degradation of at least one of the matrix forming compound and the degradable cross-linking groups.

Certain embodiments according to the present disclosure are directed to a nano-structured network hydrogel. In certain embodiments, the nano-structured hydrogel comprises a nanostructured bioconjugate and a matrix forming compound wherein the nanostructured bioconjugate and the matrix forming compound form a porous three-dimensional nano-structured network hydrogel.

In certain specific embodiments, nano-structured network hydrogel comprises a nanostructured bioconjugate, wherein the nanostructured bioconjugate comprises a nano-gel structure and a nucleic acid. In certain other specific embodiments, nano-structured network hydrogel comprises a nanostructured bioconjugate, wherein the nanostructured bioconjugate comprises a star copolymer and a nucleic acid. In certain embodiments, the nano-structured network hydrogel comprises a nanostructured bioconjugate and a matrix forming compound, wherein the matrix forming compound is selected from the group consisting of thiolated hyaluronic acid, thiolated collagen, non-thiolated collagen and combinations of any thereof. In certain embodiments, the nano-structured network hydrogel comprises a nanostructured bioconjugate and a matrix forming compound, wherein the matrix forming compound is non-thiolated collagen. In certain other embodiments, the nanostructured bioconjugate and the matrix forming compound, in the nano-structured network hydrogel, react to form the porous three-dimensional nano-structured network hydrogel connected by a plurality of degradable cross-linking connections. In certain other specific embodiments, the nano-structured network hydrogel is capable of delayed delivery of the nucleic acid by degradation of at least one of the matrix forming compound and the degradable cross-linking groups.

The thiolated hyaluronic acid (t-HA) that is incorporated into the nano-structured hydrogel as an exemplifying scaffold is a natural bioactive, biocompatible, resorbable and angiogenic polymer [Bencherif et. al., Biomacromolecules 2009, 10, (9), 2499-2507]. HA degrades enzymatically and is used in medical applications [Brekke et al., Biomedical engineering series 2005, 219-240] and plays an important role in wound healing and drug delivery [Gupta, P.; et. al., Drug Discov Today 2002, 7, (10), 569-79]. In one exemplification an in situ hydrogel is formed by the combination of the two reactive pre-hydrogel components (t-HA and an AC-PN compositionally tailored for siRNA complexation) via a Michael-type addition reaction. The t-HA and AC-PN polymer precursors of the composite are injectable and quickly react in vivo forming a hydrogel, thus permitting minimally invasive surgical applications. The release kinetics of the siRNA encapsulated in AC-PN is controlled independently of the t-HA scaffold degradation rate but the binding of the AC-PN into the t-HA network modulates the release of siRNA, thereby minimizing a burst release in the initial time period.

Certain embodiments according to the present disclosure are directed to a nano-structured network hydrogel. In certain embodiments, the nano-structured hydrogel comprises a nanostructured bioconjugate and a matrix forming compound wherein the nanostructured bioconjugate and the matrix forming compound form a porous three-dimensional nano-structured network hydrogel.

In certain specific embodiments, nano-structured network hydrogel comprises a nanostructured bioconjugate, wherein the nanostructured bioconjugate comprises a nano-gel structure and a nucleic acid. In certain other specific embodiments, nano-structured network hydrogel comprises a nanostructured bioconjugate, wherein the nanostructured bioconjugate comprises a star copolymer and a nucleic acid. In certain embodiments, the nano-structured network hydrogel comprises a nanostructured bioconjugate and a matrix forming compound, wherein the matrix forming compound is selected from the group consisting of thiolated hyaluronic acid, thiolated collagen, non-thiolated collagen and combinations of any thereof. In certain embodiments, the nano-structured network hydrogel comprises a nanostructured bioconjugate and a matrix forming compound, wherein the matrix forming compound is non-thiolated collagen. In certain other embodiments, the nanostructured bioconjugate and the matrix forming compound, in the nano-structured network hydrogel, react to form the porous three-dimensional nano-structured network hydrogel connected by a plurality of degradable cross-linking connections. In certain other specific embodiments, the nano-structured network hydrogel is capable of delayed delivery of the nucleic acid by degradation of at least one of the matrix forming compound and the degradable cross-linking groups. For example, after in vivo formation of the nano-structured network hydrogel at the site, the structure of the network hydrogel may slowly degrade, for example by degradation of the degradable linking groups within the polymeric nanostructure and/or degradation of the links between the matrix forming compound. As the degradable linking groups within the polymeric nanostructure degrade, the nucleic acid, for example, the siRNA, is released from the structure and can be delivered to the active site in the cell. As the links in the matrix degrade, the overall matrix slowly degrades and is ultimately flushed from the patient's system during the natural course of time.

In one embodiment, functionalization of the polymers with ligands, antibodies or short peptides, exemplified herein by fibronectin fragments; H-Gly-Arg-Gly-Asp-Ser-OH (GRGDS), that are incorporated into the primary chain via use of functional initiators, exemplified by a N-hydroxysuccinimide-PEO (NHS-PEO) with a bromoisobutyrate groups, or through co-monomers, such as 2-hydroxyethyl methacrylate (HEMA), 2-hydroxyethyl acrylate (HEA) that are subsequently functionalized or by displacement of terminal bromine with azides, and subsequent “click” reaction with moieties containing complementary alkyne groups [Siegwart, D. J.; et. al., Macromol. Chem. Phys. 2008, 209, (21), 2179-2193] for cell targeted delivery. The core of the stars of nanogels are made cationic by incorporating either amino or ammonium groups via co-polymerization with either 2-dimethylaminoethyl methacrylate (DMAEMA) or quaternized DMAEMA (qCDMAEMA) to bind specific negatively charged siRNA's providing encapsulation, stability and customizable pharmacokinetic release [Merkel, O. M. et. al., J. Controlled Release 2009, 138, (2), 148-159]. One procedure for encapsulation of biomolecules is accomplished by swelling the AC nanogel [Oh, J. K.; et. al., Prog. Polym. Sci. 2008, 33, (4), 448-477] in the presence of the selected siRNA. The t-AC-PN is functionalized with acrylate groups and subsequently cross linked via Michael addition with t-HA.

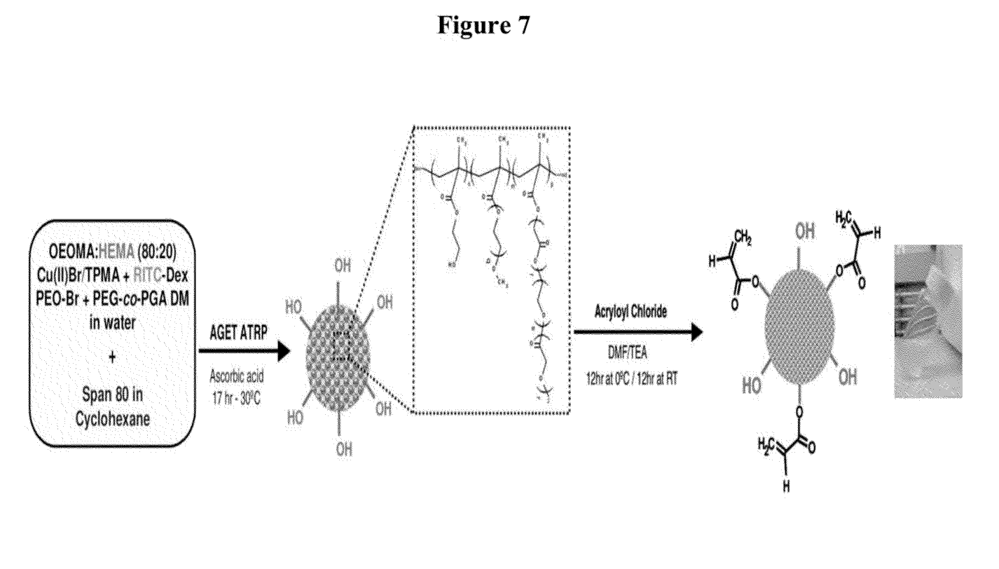

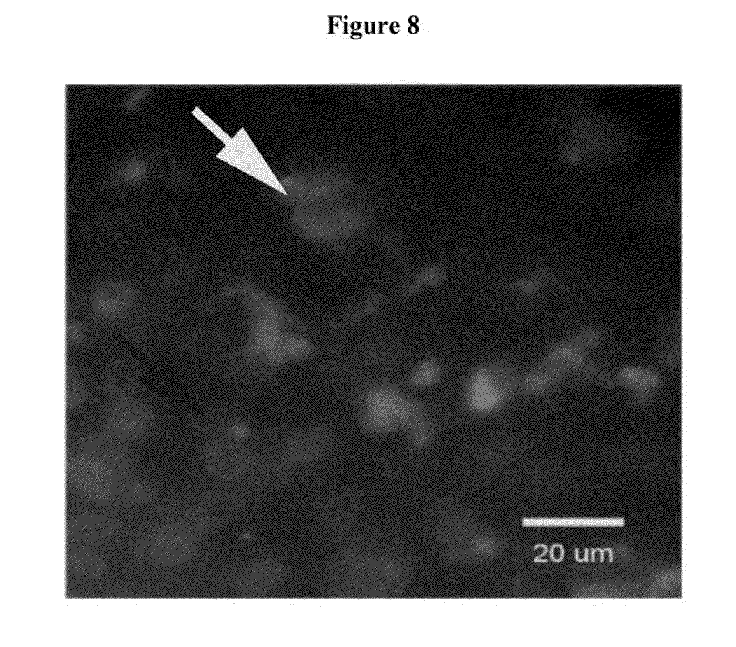

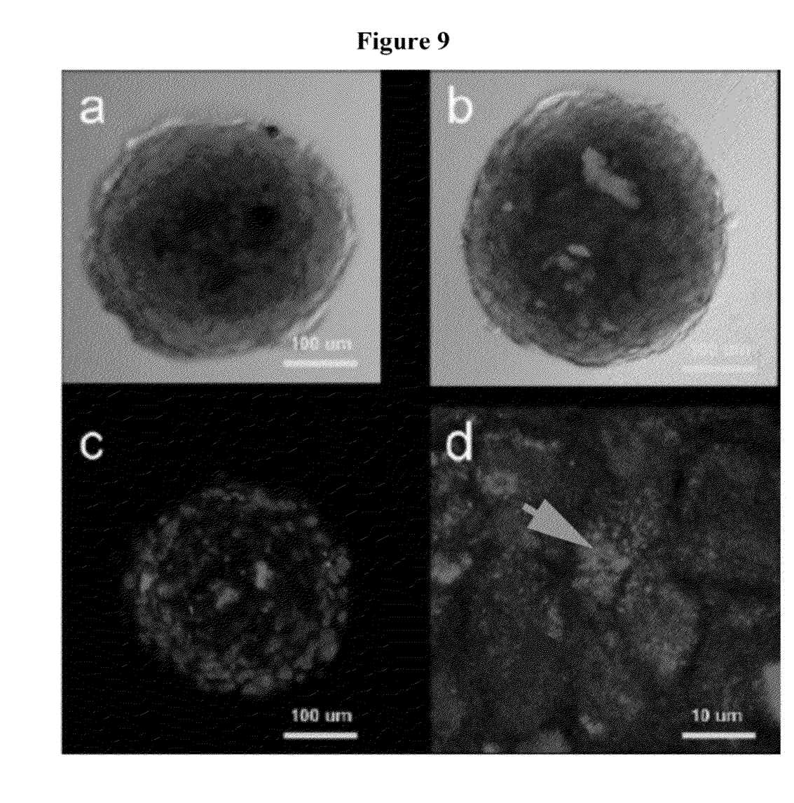

To exemplify degradation of RITC-Dx loaded POEO300MA-co-PHEMA nanogels synthesized by inverse mini emulsion with an entrapped fluorescent dye (FIG. 7) are well-dispersed in water and degrade in water through the acid-catalyzed hydrolysis mechanism. The average diameter of the nano-structured polymers decreased from 203 nm to 9 nm corresponding to the size of individual polymeric sols from degradation. Nanogels with uniform size are biocompatible and can be delivered into cells. This is exemplified by synthesizing nanogels via ATRP copolymerization of OEO₃₀₀MA and 2-hydroxyethyl methacrylate (HEMA) with degradable poly(glycolic acid) (PGA) cross linkers and loading with a fluorescent dye and bovine serum albumin (BSA) or FITC-Dx. MC3T3-E1.4 osteoblast cells are transfected with the BSA protein loaded nanogel and demonstrate the utility of the AC nanogel-FITC-Dextran (Dx) internalization into complex co-culture system comprising human umbilical vein endothelial cells (HUVEC) and human mesenchymal stem cells (hMSC). Nanogels encapsulated with BSA protein are measured within 6 hours after incubation within MC3T3-E1.4 cells, (FIG. 8) suggesting early entry into the cells. FITC-Dx loaded nanogels penetrate 3D spheroidal co-cultures of hMSC and HUVEC (FIG. 9). In one example, an aqueous-dispersable AC-nanogel is prepared via ATRP in an inverse mini emulsion and the nanogels degraded to individual non-toxic polymeric chains. Uptake of nanogels with BSA and fluorescent dye is observed as early as 1 hour in the HUVEC/hMSC co-culture cells suggesting nanogels can be effectively used for delivery of functional biomolecules (e.g., BMP's and siRNA's).

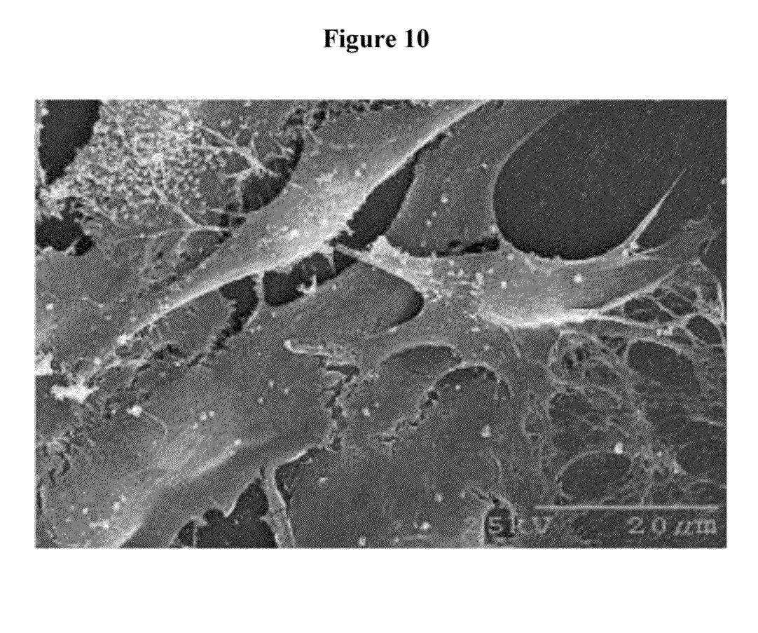

Biocompatible nano-structured star polymers, composed of poly(ethylene oxide) (PEO) arms with incorporated tethered GRGDS moieties that facilitate cell attachment, are synthesized via ATRP of the first formed macromonomer in the presence of divinyl compounds and optional functional mono-vinyl monomer(s). In vitro cytotoxicity experiments reveal the nano-structured star polymers to be biocompatible (≧95% cell viability). Scanning electron microscopy (SEM) reveals that uniform cell adhesion and distribution (FIG. 10) is promoted by GRGDS functionalized star nano-structured polymers. The uncoated surface does not show any cell attachment [Bencherif, S. A.; et. al., Biomacromolecules 2009]. Data confirms that the crosslinked PEO nano-structured star polymers are biocompatible and can be functionalized to target cell specific receptors, which is a key for delivery of selected siRNA's to targeted cells.

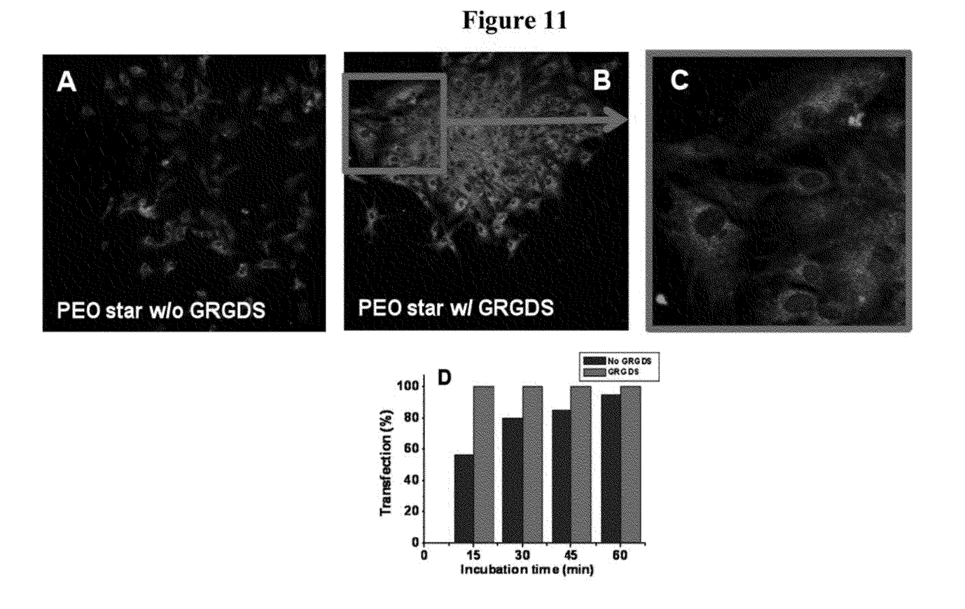

In one embodiment, the functional nano-structured PEO star polymers are tailored to transfect MC3T3-E1.4 pre-osteoblast cells. The purpose of this exemplification is to determine internalization efficiency of nano-structured star polymers with and without tethered GRGDS. PEO star polymers with and without GRGDS show internalization in MC3T3-E1.4 pre-osteoblast cells (FIG. 11). Flow cytometry analysis shows 100% cell internalization with RGD conjugated nano-structured star polymers as early as 15 min. when compared to nano-structured polymers without RGD, indicating rapid cellular uptake of GRGDS functionalized star nano-structured polymers by cells. The fluorescent FITC conjugated PEO star polymers reveal efficient internalization with GRGDS when compared to nano-structured polymers without GRGDS suggesting functionalized nano-structured materials will have rapid and high internalization efficiency (100%) when compared to non-functionalized nano-structured materials.

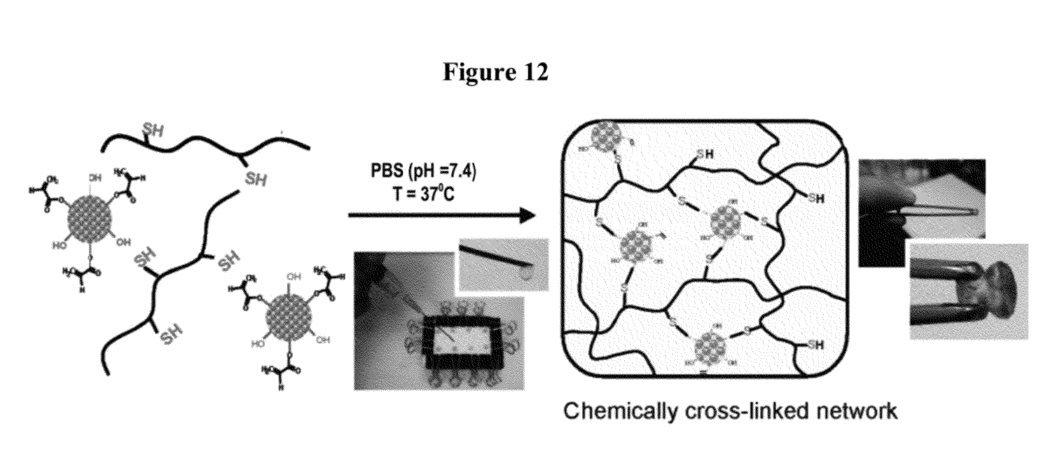

In one embodiment, mechanically stable in situ (for example, in-vivo) nano-structured hydrogels are prepared with a hyaluronic acid backbone and polymeric nanoparticles acting as crosslinks. The in situ nano-structured hydrogels are formed from the combination of two reactive pre-hydrogel components, i.e., the nanostructured bioconjugate and the matrix forming compound. The nanogels are uniformly distributed in the porous three-dimensional (3D) structure of the formed hydrogel matrix. Uniform cross-linked networks of t-HA-Nanogels are synthesized with customized form and predictable mechanical properties via AGET ATRP of OEO300MA and HEMA in inverse miniemulsion of water/cyclohexane (FIG. 12). Surface morphology demonstrates that the nano-structured gel has a porous three-dimensional (3D) structure and uniform distribution of nanogels within the scaffold.

Indeed the scaffold may play a key role as a material component involved in successful preventative treatment of HO. The scaffold provides critical elements of function to the overall composite material (i.e., scaffold+nanomaterial) by providing: (1) mechanical integrity for facile surgical implantation, (2) sequestration, transport, and containment of embedded nano-structured materials, (3) biocompatibility, (4) degradation and absorption, and (5) controlled release of cargo material (i.e., siRNA) over the desired window of treatment. The application describes the critical elements of function in terms of biological performance criteria. Significant thought was required to develop a suitable material to address each of the performance criteria. In one embodiment of the invention collagen is selected as the scaffold material. Type I collagen has FDA approval and t-HA does not; it is biocompatible and presents an ease of synthetic preparation and fabrication into a suitable scaffold material superior to the t-HA scaffold in that it enjoys FDA approval and injectable collagen scaffolds are known. [Hartwell, R., et al. Acta Biomater. 2011. 7(8): 3060-3069].

In other embodiments, collagen may be used as the matrix forming compound to form the scaffold with the nanostructured bioconjugate. The collagen/nanostructure bioconjugate may be synthesized ex vivo and then installed within the patient via a surgical process. In order to synthesize the collagen scaffold in a highly reproducible fashion to exemplify the utility of collagen in this procedure, a Teflon/steel mold was designed and fabricated since initial attempts to fabricate scaffolds without a mold were unacceptable and produced an inconsistent variable scaffold product. Numerous challenges were overcome and addressed in the early stages of scaffold fabrication and accomplishments were made in terms of collagen production:

(1) achievement of super concentrated solutions of collagen (ca. 22 mg/ml),

(2) uniform and consistent scaffold structures,

(3) mechanical integrity,

(4) efficient solution transfer protocols of highly viscous collagen solutions and

(5) effective methods for removal of air pocket and voids.

The designed mold provided improved reproducibility and many desirable characteristics. These characteristics include a uniform and efficient compression mechanism, protein scaffold adhesion prevention (i.e., to the Teflon material), high throughput capabilities, guided pin scaffold ejection, and simple assembly/disassembly. A uniform compression force is required to retain sample solutions in their respective cavities and is accomplished using top and bottom compression plates. The dimensions of the mold cavities were selected based upon a benchmark collagen composite scaffold. The commercially available collagen scaffold from Becton Dickenson (Franklin Lakes, N.J.) was used as a suitable model. Composite structures of different topology would be required for insertion by minimally invasive surgery but deposition at certain sites in the body may require an open surgical approach.

Once a successful scaffold was obtained from a macroscopic perspective, further characterization was completed to confirm the microstructure and homogeneity of the scaffold. Environmental scanning electron microscopy (E-SEM) images were obtained for the exterior surface and interior cross section to elucidate the structure of the scaffold. Generally, the scaffold structure shows similar morphology in both the interior and exterior. However, small differences in density/porosity exist at low magnification with the exterior exhibiting a slightly more dense shell in comparison to the interior of the scaffold. Overall, the SEM images verify that the structure of the scaffold is homogenous in nature with uniform interconnected pores, without the presence of large voids from residual air pockets. The interconnect pore structure is clearly visible under high magnification. Further characterization of the collagen scaffold was completed to confirm purity and absence of undesirable contaminates during the molding process. Energy-dispersive X-ray spectroscopy (EDX) was used to confirm the relative concentrations of elements contained within the scaffold. As expected, the scaffold material was composed solely of carbon, nitrogen, and oxygen. Injectable and biodegradable hydrogel scaffolds hybridized with nanogels are produced. The chemistry offers the advantage of selective, in situ crosslinking and functions as a biocompatible matrix for cell and protein encapsulation and delivery of bioactive molecules, e.g., the siRNA's.

Certain embodiments according to the present disclosure are directed to a method of treating a clinical condition comprising: administering to a patient having a clinical condition a nanostructured bioconjugate, administering to the patient a matrix forming compound; and forming a nano-structured network hydrogel in-vivo, wherein the nano-structured network hydrogel and the nanostructured bioconjugate degrade releasing the nucleic acid to a localized targeted biological site. In other embodiment, the nanostructured bioconjugate and the matrix forming compound may be reacted ex vivo and then administered to the patient via an injection or surgical procedure.

In certain embodiments, a different ratio of the nucleic acid in the nanostructured bioconjugate and matrix forming compound is used.

Various features of the present invention will become more apparent upon consideration of the following examples. The various embodiments of this disclosure described in the following examples are not to be considered as limiting the invention to their details. All parts and percentages in the examples, as well as throughout this specification, are by weight unless otherwise indicated.

### EXAMPLES AND DISCUSSION OF EXAMPLES

### Abbreviations

AC-PN Acrylated cationic polymeric nanomaterials
ACVR1 Activin A receptor type 1
Adex-Cre Adenovirus that expresses Cre recombinase
ALK2 Activin receptor-like kinase 2
ATRP Atom transfer radical polymerization
BMP Bone morphogenetic protein
C2C12 Murine myoblast cell line
CRP Controlled radical polymerization
CS Core shell
DMAEMA: 2-(dimethylamino)ethyl methacrylate
DMF Dimethylformamide
EGDMA Ethylene glycol dimethacrylate
EO Ectopic ossification
FGF Fibroblast growth factor
FITC-Dx Fluorescein isothiocyanate-dextran
GPC Gel permeation chromatography
GRGDS Glycine-Arginine-Glycine-Aspartate-Serine
GSH Glutathione
HEA 2-hydroxyethyl acrylate
HEMA 2-hydroxyethyl methacrylate
hMSC Human mesenchymal stem cells
HMTETA 1, 1, 4, 7, 10, 10-hexamethyl triethylenetetramine
HO Heterotopic ossification
HUVEC Human umbilical vein endothelial cells
INT (2-(4-iodophenyl)-3-(4-nitrophenyl)-5-phenyl tetrazolium chloride)
LDH Lactate dehydrogenase
LLC-PK1 Porcine kidney epithelial cell line
MC3T3-E1.4 Murine calvarial pre-osteoblast like, embryonic day 1 subclone 4 cells
MTS 3-(4,5-dimethylthiazol-2-yl)-5-(3-carboxymethoxyphenyl)-2-(4-sulfophenyl)-2H-tetrazolium
NG Nanogel
NHS-PEO N-hydroxysuccinimide-polyethylene oxide
NMP Nitroxide mediated polymerization
NSAID Non-steroidal anti-inflammatory drug
NSP Nano-structured polymer
OEOMA Oligoethyloxide methacrylate
Osx Osterix
PDGF Platelet derived growth factor
PEG Poly(ethylene glycol)
PEGMA Poly(ethylene glycol) methacrylate
PEI Poly(ethylene imine)
PGA Polyglycolic acid
PMMA Poly(methyl methacrylate)
PTH Parathyroid hormone
QDMAEMA Quaternized 2-(dimethylamino)ethyl methacrylate
Q-PCR Quantitative polymerase chain reaction
RAFT Reversible additional fragmentation chain transfer
RI Refractive index
RISC RNA induced silencing complex
Runx2 Runt related protein 2
siRNA Short interfering ribonucleic acids
SSX bis(2-methacryloyloxyethyl) (disulfide crosslinker)
TGF-b Transforming growth factor-beta
TRITC Trimethyl rhodamine isothiocyanate
UV Ultraviolet

To exemplify the ability to selectively prepare bioconjugates for delivery of nucleic acids (RNA/DNA) targeting symptomatic and asymptomatic oncological and pathological disease a series of nano-structured polymers (NSPs) were synthesized for evaluation in preventative treatment of HO. The first formed materials for selective delivery of siRNA were cationic star copolymers and nanogels of differing dimensions were synthesized. A standard nomenclature, described above, was developed that will utilize to describe the nano-structured polymer portfolio developed to exemplify the utility of the invention. Cationic star polymers are the smallest in size, with a typical diameter of 10-50 nm. This size allows for fast cellular internalization, but also limits siRNA loading capacity. Core-shell nanogels are slightly larger, with a diameter between 100-200 nm, a size expected to lower internalization kinetics, but providing an increase in loading capacity. Cationic nanogels are the largest NSPs, with a diameter between 100-300 nm. While internalization would be comparatively slower than the former polymers, the 100-300 nm size should have the highest siRNA loading capacity.

A variety of -nano-structured copolymers are prepared to determine the concept and illustrate the ability to prepare materials that can control delivery of nucleic acids from cationic nano-structured bioconjugates that selectively target multiple symptomatic or asymptomatic ‘clinical entities’ to targeted cells herein exemplified by delivery of preselected siRNA's to stop heterotopic ossification. As illustrated below the gross size of the nano-structured delivery vehical is one method to control delivery rate and release kinetics another is the intrapparticle architecture which can be modified/adjusted to increase adsorption and release of different nucleic acids.

### Example 1

The example describes the preparation of cationic star copolymers with degradable cores prepared through the addition of 2-(dimethylamino)ethyl methacrylate (DMAEMA) and bis(2-methacryloyloxyethyl) disulfide cross-linker (SSX) to PEO macroinitiators in an arm first synthetic approach. The cationic star polymers were assessed for biocompatibility using human mesenchymal stem cells (hMSCs), porcine kidney epithelial cell lines (LLC-PK1), mouse calvarial pre-osteoblast like cells, embryonic day 1 subclone 4 cells from the MC3T3-E1.4 cell line and murine myoblast cells (C2C12). Furthermore, the biodegradation of the star copolymers prepared with SSX in the presence of glutathione (GSH) under conditions mimicking a reductive biological environment was successful.

1 A) (PEG)ₙ-Poly(DMAEMA-co-SSX) Star Polymers were Synthesized Via an “Arm-First” Method.

The ratio of reagents used was: [PEGMA]₀/[DMAEMA]₀[SSX]₀[EBiB]₀/[CuCl/HMTETA]₀=1/0.5/1/0.21/0.42. PEGMA (Mₙ=2080, 2.0 g, 1.0 mmol), DMAEMA (78.6 mg, 0.5 mmol), SSX (290.4 mg, 1.0 mmol), HMTETA (114.2 μL, 0.42 mmol), and methanol (20 mL) were charged to a Schlenk flask. The flask was degassed by five freeze-pump-thaw cycles and filled with nitrogen. CuCl (42.0 mg, 0.42 mmol) was quickly added to the frozen mixture under nitrogen. The flask was sealed with a glass stopper then immersed in a 60° C. oil bath. The deoxygenated initiator EBiB (31.0 μL, 0.21 mmol) was injected into the reaction system via a nitrogen purged syringe. At pre-defined time intervals, samples were withdrawn via a syringe and immediately diluted with DMF for analysis. The samples were used to measure polymer molecular weights by GPC. The reaction was stopped after 72 h by exposure to air. The final star polymers were purified by dialysis against methanol and distilled water for 2 days, respectively, by using a dialysis bag with molecular weight cutoff (MWCO)=25000. The compositional results are presented in Table 2.

TABLE 2
Preparation of Star Polymers via an ATRP “Arm-First” Method^{a1}

| Entry | Crosslinker | M_{n,RI}^{b} × 10⁻³ | M_{w}/Mₙ^{c} | M_{w,MALLS}^{d} × 10⁻³ | Nₐᵣₘ^{e} | Dₕ^{f} (nm) |
|---|---|---|---|---|---|---|
| D-SSX^{g} | SSX | 41.0 | 1.71 | 91.1 | 50 | 12.4 ± 0.9 |
| QD-SSX^{h} | SSX | 44.2 | 1.55 | 90.3 | 49 | 12.1 ± 0.9 |
| D-EGDMAⁱ | EGDMA | 47.1 | 1.33 | 94.7 | 53 | 10.4. ± 0.9 |

^{a}Experimental conditions: [PEGMA]₀ = 0.05 M at 60° C. in methanol, stopped at 72 h.
^{b}Number-average molecular weight, measure by DMF GPC with RI detector, calibrated with linear PMMA standards.
^{c}Polydispersity, measured by DMF GPC with RI detector, calibrated with linear PMMA standards.
^{d}Weight-average molecular weight, measured by THF GPC with MALLS detector.
^{e}Number-average value of the number of arms per star molecule.
^{f}Hydrodynamic volume in H₂O, measured by DLS and size expressed as D_{avg} ± SD (average diameter ± standard deviation)
^{g}[PEGMA]₀/[DMAEMA]₀/[SSX]₀/[EBiB]₀/[CuCl/HMTETA]₀ = 1/0.5/1/0.21/0.42.
^{h}[PEGMA]₀/[QDMAEMA]₀/[SSX]₀/[EBiB]₀/[CuCl/HMTETA]₀ = 1/0.5/1/0.21/0.42.
ⁱ[PEGMA]₀/[DMAEMA]₀/[EGDMA]₀/[EBiB]₀/[CuCl/HMTETA]₀ = 1/0.5/1/0.21/0.42.

Incorporation of cationic monomers DMAEMA or QDMAEMA into the core of the star polymers facilitated electrostatic complexation with the negatively charged siRNA. The initial zeta potential of NSP-S-SSX-5% N and NSP-S-SSX-5% QN was 22.5±1.32 mV and 2.38±0.73 mV while the zeta potential of NSP-S-SSX-0% N was −6.68±1.90 mV because of different core charges. The zeta potential value of NSP-S-SSX-5% N decreased from 22.9±1.78 mV to −26.3±1.27 mV with increasing incorporation of siRNA (with degreasing N/P ratio from 10 to 0.2). The variation of zeta potential on N/P ratio is smaller in the case of NSP-S-SSX-5% QN, indicating the efficacy of siRNA complexation of NSP-S-SSX-5% N is lower than for NSP-S-SSX-5% QN. The hydrodynamic volume of the NSP-S complexed with siRNA did not change significantly with N/P ratios (for example, hydrodynamic diameter of NSP-S-SSX-5% QN varied from 12.53±1.00 to 12.82±1.00 nm for N/P=0.2 and 10, respectively), suggesting siRNA complexed star polymers remained as individual polymers without aggregation.

### 1 B) Degradation of Star Polymers.

The core structure of cationic NSP-S contains degradable disulfide cross-linker, which is cleaved under in vivo reducing conditions (i.e., in the presence of glutathione (GSH). GSH is a natural molecule present in the body. The rationale for polymers with disulfide-functionalized cross-linkers is to produce biomaterials that undergo controlled biodegradation (e.g., by natural endogenous GSH) and thus will have predictable biological performance properties as therapeutics. Moreover, the disulfide bond is naturally present in a majority of proteins and enzymes and is cleaved in vivo in the presence of GSH, the most abundant intracellular thiol.

Star polymers were dispersed in DNase/RNase-free distilled water without additional surfactants and mixed with a glutathione solution. The final star polymer and glutathione (GSH) concentration was 2 mg/mL and 100 mM, respectively. The solution was incubated with 5% CO₂ at 37° C. for 48 h. The variation in the hydrodynamic diameter of NSP-S_{SSX}-C and NSP-S_{SSX}-qC was measured by dynamic light scattering (DLS). DLS analysis of the hydrodynamic diameter of NSP-S_{SSX}-C and NSP-S_{SSX}-qC after 48 h incubation with 100 mM GSH solution showed a decrease in particle size suggesting that the core of the star polymers was degraded into individual polymeric chains. Control studies were preformed in the absence of GSH and showed no degradation of the star polymer. For a negative control experiment, non-degradable NSP-S_{EGDMA}-C was tested under the same conditions as NSP-S_{SSX}-C and NSP-S_{SSX}-qC. The size distribution of NSP-S_{EGDMA}-C with 100 mM GHS was similar to its control group (0 mM GSH treatment). This outcome indicated the EGDMA cross-linker is non-degradable on GSH and validates that the SSX crosslinker is required for degradation.

Degradation of NSP-S was determined under physiological GSH concentration (10 mM) in a nitrogen atmosphere at room temperature. DLS analysis of NSP-S-SSX-5% N and NSP-S-SSX-5% QN underscored successful degradation in 4 days, whereas NSP-S-SSX-0% N completely degraded after 6 days of incubation.

TABLE 3
Degradation under a physiological GSH
concentration (10 mM) analyzed with DLS^{a}

| | Before Degradation | After Degradation | Time |
|---|---|---|---|
| Entry | (nm) | (nm) | (days) |
| NSP-S-SSX-5% N | 10.3 ± 2.07 | 0.97 ± 0.22 | 4 |
| NSP-S-SSX-5% QN | 15.2 ± 1.58 | 0.97 ± 0.15 | 4 |
| NSP-S-SSX-0% N | 14.5. ± 1.68 | 1.00 ± 0.15 | 6 |

^{a}Polymer concentration was 0.25 mg/mL with DNase/RNase free water under N₂ at room temperature.

NSP-S-SSX-5% N and NSP-S-SSX-5% QN compared to NSP-S-SSX-0% N have less compact cores due to the incorporation of cationic monomers. This may affect the rate of degradation under the same conditions (Table 3). The ability to control and vary different core densities allows the design of materials with tunable degradability, release kinetics properties, and biocompatibility.

### 1 C) In Vitro Biocompatibility of Star Polymers.

Three star polymers, with different physico-chemical properties, were used in the first set of experiments to determine the biocompatibility of the star polymers: NSP-S_{SSX}-N, NSP-S_{SSX}-C and NSP-S_{SSX}-qC. The neutrally charged star, NSP-S_{SSX}-N, is the prototype of the star polymers that were modified by adding either 5 wt % DMAEMA or 5 wt % qDMAEMA to the polymer core, giving it cationic properties that allow for complexation with anionic siRNA. The experiments were designed to compare the cytotoxicity of each type of star polymer. Four different cell types were used:

1) human mesenchymal stem cells (hMSCs) (Lonza PT2501).

2) porcine kidney epithelial cell line (LLC-PK1) (ATCC CL-101)

3) murine calvarial preosteoblast cell line (MC3T3-E1.4) (ATCC CRL-2593)

4) murine myoblast cell line (C2C12) (ATCC CRL-1772).

HMSCs, MC3T3-E1.4 and C2C12 cells are osteoprogenitor cells or cell lines. These cells may reflect the process of osteogenesis during heterotopic ossification. LLC-PK1 cells are suggested by American Society for Testing and Materials (ASTM) E2526-08 as part of a standard method for the evaluation of cytotoxicity of nanoparticulate materials.

Biocompatibility of the star polymers was determined by ASTM and ISO standards. Three different assays were performed, each covering a specific aspect of the biocompatibility criteria. Using multiple assays provides a more comprehensive understanding of these polymers. The logic for each assay is stated in the background of each of the three assays.

The three assays include:
1. 3-(4,5-dimethylthiazol-2-yl)-5-(3-carboxymethoxyphenyl)-2-(4-sulfophenyl)-2H-tetrazolium assay (MTS) to determine cell viability
2. Lactate Dehydrogenase assay (LDH) to assess cellular cytotoxicity
3. PicoGreen assay to determine cellular proliferation.

### MTS Assay

MTS is a colorimetric assay that determines cell viability by measuring the activity of mitochondrial enzymes. Viable cells retain the necessary enzymes needed to convert 3-(4,5-dimethylthiazol-2-yl)-5-(3-carboxymethoxyphenyl)-2-(4-sulfophenyl)-2H-tetrazolium (MTS) into a formazan product. The amount of formazan produced is measured via absorbance.

A CellTiter 96® Aqueous One Solution Assay (Promega G3580) was utilized to measure formazan production. The cells used were hMSCs, LLC-PK1 cells, MC3T3-E1.4 cells, and C2C12 cells. Cells were seeded at a density of 10,000 cells/mL in a 48 well plate (Costar 3548). These were cultured for a period of at least 48 hours. Exposure to polymer was initiated once the cells had reached 80% confluency. Media containing concentrations of 100, 200, 400, and 800 μg/mL of the star polymers were added to the wells. After culturing for an additional 48 hours, media was collected in 1.5 mL eppendorf tubes (Fisherbrand 02-681-284), and 200 μL of that returned to its respective well on the 48 well plate. The plates were then incubated at 37° C. and 5% CO₂ for another 60 minutes to allow the pH of the media to stabilize. Once completed, 40 μL of MTS reagent was added to each well and incubated at 37° C. and 5% CO₂ for 20-30 minutes. The absorbance was read at 492 nm using a Tecan Spectra Fluor, and the data recorded in terms of optical density, or OD, value. The percentage of cell viability was normalized to cells alone, which was set to 100% cell viability. Percentage cell viability was calculated using the

$$\% Cell Viability = \frac{{OD}_{sample} - {OD}_{media}}{{OD}_{cells} - {OD}_{media}}$$

Polyethyleneimine (PEI) (Sigma 408727-250ML) in concentrations of 100, 200, 400, and 800 μg/mL was used as a negative control. A decrease in cell viability was expected even though PEI is a polymer commonly used for siRNA delivery, it is reported to be highly cytotoxic due to its cationic nature. A cells alone group (no treatment) served as the positive control, in which high cell viability was expected.

### Results

The star polymers were biocompatible. A slight suppression in cell viability was observed for hMSCs. However, there was no statistical difference between the rest of the experimental cohort and the positive control. Results were recorded as the mean±a standard deviation and sample size for each group was three (n=3). The nanogel polymers did not induce elevated levels of LDH release. There was no statistical difference between the experimental cohort and the negative control. All data was evaluated as mean±standard deviation and the sample size for each group was three (n=3). MTS Results for hMSC after 48 hours exposure indicated that mitochondrial activity appeared to have decreased by roughly 20% when the star polymers were added to hMSCs. However, there was no significant difference in cell viability among the three star polymer treatment groups. The addition of PEI to cells suppressed enzyme activity by 80%.

The cytotoxicity data from the MTS and LDH assays indicate that the NSP-S is biocompatible. MTS results for LLC-PK1 after 48 hours exposure showed there was no significant difference in cell viability between cells alone, and cells with star polymer treatment. Once again, PEI had a significant impact on cell viability, which was decreased by approximately 70%.

Data from MTS results for MC3T3-E1.4 after 48 hours exposure suggested the neutrally charged star polymer decreased cell viability as concentration was increased, but this trend was not observed for the NSP-S_{SSX}-C and NSP-S_{SSX}-qC star polymers. PEI decreased cell viability by 60-70%.

Star polymers decreased cell viability by up to 20% for the neutral type, but only 10% for the cationic types (5% DMAEMA and 5% qDMAEMA). PEI induced an 80% decrease in cell viability.

### LDH Assay

LDH is a colorimetric assay that measures cytotoxicity by quantifying the lactate dehydrogenase (LDH) activity in the media. LDH is a membrane enzyme that is leaked into the media when the cell membrane is damaged as a result of cytotoxic biomaterials. LDH converts NAD⁺ to NADH, which when added to the tetrazolium salt (2-(4-iodophenyl)-3-(4-nitrophenyl)-5-phenyl tetrazolium chloride) (INT), converts it to a red formazan product.

The cell types used for this study were the hMSC, LLC-PK1, MC3T3-E1.4, and C2C12 cells. LDH levels were measured using the CytoTox 96® Non-Radioactive Cytotoxicity Assay (Promega G1780). Media samples collected for LDH analysis were from the same wells as that used for the MTS. 100 μL of cell lysis buffer (Cell Signaling 9803) was added to the lysis group 45 minutes before media collection. The media was then centrifuged at 2000 rpm for 5 minutes to remove any cells in the media, and the supernatant transferred to a new eppendorf tube (Fisherbrand 02-681-284). All samples were centrifuged again at 13000 rpm for 7 minutes to remove nanoparticles. The supernatant was then split into three 200 μL aliquots, and frozen at −80° C. until ready for use. To run the LDH assay, samples were first thawed at 37° C., and 50 μL of each were added to a transparent 96-well plate (Fisherbrand 12-565-501). This was followed by adding 50 μL of LDH substrate, incubating the plate for 30 minutes at room temperature, and adding 50 μL of stop solution. The absorbance was read at 492 nm with a Tecan Spectra Fluor and the results recorded. The percent of LDH released was calculated using the following formula:

$$\% LDH Released = \frac{{OD}_{sample} - {OD}_{media}}{{OD}_{lysis} - {OD}_{media}}$$

PEI (Sigma 408727-250ML) at concentrations of 100, 200, 400, and 800 μg/mL was used as a positive control since was expected to cause an increase in LDH release, indicating cytotoxicity. A cells alone cohort (without treatment) served as the negative control since minimal LDH release was expected.

### Results

The star polymers did not induce elevated levels of LDH release. There was no statistical difference between the experimental cohort and the negative control. Data was reported as mean±standard deviation and the sample size for each group was three (n=3). The LDH results for hMSCs after 48 hours exposure showed no significant increase in LDH when star polymers were added compared to the cells alone control; whereas PEI induced up to 65% LDH release. The LDH results for LLC-PK1 after 48 hours exposure indicated that minimal amounts of LDH, if any, were released for the cells alone of cells in the presence of star polymer groups. However, PEI treatment resulted in up to 60% LDH release. LDH results for MC3T3-E1.4 after 48 hours exposure also showed the presence of star polymers continued to have no significant effect on LDH release, but PEI caused up to 52% LDH release. LDH results for C2C12 after 48 hours exposure also demonstrated that star polymers and cells alone had comparable amounts of LDH release. PEI had significantly higher levels with up to 33% LDH released.

### PicoGreen Assay

PicoGreen is a fluorescent assay that quantifies double stranded DNA concentration using a nucleic acid stain PicoGreen (Invitrogen P7589). DNA concentration is directly proportional to cell number. Consequently, the PicoGreen is an indicator of cell proliferation. Cells were cultured in 24 well tissue culture plates (Costar 3524) for hMSCs, and in 12 well tissue culture plates (Costar 3513) for MC3T3-E1.4 cells over a period of 10 days. Polymers were added (100 μg/mL for star polymers, 1 μg/mL for PEI) to cells 48 hours after seeding, and incubated at 37° C. and 5% CO₂. The media was changed every 48 hours, with the same concentration of polymer added to the new media each time. Samples were collected at 0, 4, 7, and 10 days of treatment. At each time period, the media was removed and 600 μL of 1× cell lysis buffer (Cell Signaling 9803) added to each of the wells. The plates immediately went through three freeze-thaw cycles (each cycle involves incubation at −80° C. for 20 minutes, followed by incubation at 37° C. for 20 minutes). Samples were transferred to 1.5 mL eppendorf tubes (Fisherbrand 02-681-284) and centrifuged at 13000 rpm for 7 minutes to remove debris. The contents of each tube were then transferred to two eppendorf tubes, and frozen at −80° C. until ready for use. The final volume of PicoGreen solution needed was determined by calculating the total number of wells to be used. Concentrated PicoGreen was then diluted 200-fold to form the final volume needed. A standard DNA curve was derived by diluting a concentrated DNA standard with 1× cell lysis buffer (Cell Signaling 9803) to form what would become concentrations of 0, 31.25, 62.5, 125, 250, 500, and 1000 ng/mL of DNA. Experimental samples were thawed at room temperature. Once this was completed, 100 μL of each DNA standard, as well as the experimental samples were added to a 96-well plate (Fisherbrand 12-565-501). Following this, 100 μL of diluted PicoGreen solution was added to each well, and incubated for 3 minutes. The fluorescence was then read at fluorescein excitation (485 nm) and emission (535 nm) wavelengths using a Tecan Spectra Fluor. A standard curve was created with the OD values of the DNA standards and a linear correlation determined between OD value and DNA concentration. This equation was then used to convert the OD value for each sample to DNA concentration. PEI (Sigma 408727-250ML) was used as a negative control since no proliferation was expected. A cells alone group (no treatment) served as the positive control since continuous cell proliferation was expected.

### Results

The star polymers seemed to cause a slight suppression in normal hMSC proliferation compared to the positive control. However, this phenomenon was not observed with MC3T3-E1.4 cells, where no significant difference existed between cell proliferation of cells alone and cells with star polymer treatment. PicoGreen results for hMSCs after 10 days showed that cells that had received star polymer treatment experienced a slight suppression in proliferation at days 7 and 10 compared to cells alone. While picoGreen results for MC3T3-E1.4 after 10 days showed that the star polymers had minimal, if any significant effect on MC3T3-E1.4 proliferation compared to cells alone. PEI immediately hindered cell proliferation and fully suppressed proliferation.

1 D) siRNA Complexation with Star Nano-Structured Polymers

The purpose of these experiments was to determine the siRNA complexation with star polymers by dynamic light scattering (DLS) and zeta (ζ) potential. The results will be used for down-selection from the three types of star polymers that are currently being tested.

Zeta Potential and Hydrodynamic Volume Measurement of siRNA Complexation

Fluorescein isothiocyanate (FITC) and Cyanine 3 (Cy3) conjugated fluorescent siRNAs were obtained from Santa Cruz Biotechnologies Inc. (Santa Cruz, Calif.), re-suspended to form 125 μL of stock siRNA(F) solution, and then diluted with 875 μL of sterile, nuclease-free water to form a final concentration of 2.5 mM siRNA(F). This solution was then added to dry star polymers (2 mg) and mixed overnight at 4° C. Using a dialysis membrane (Spectra/Por Biotech Regenerated Cellulose Dialysis Membranes) with MWCO=50000 the siRNA-star polymer polyion complex was separated from unbound siRNA(F) after 24 h dialysis at 4° C. siRNA(F) complexation with the cationic star polymer was determined using a Zetasizer Nano under sterile, RNAase/DNAase free conditions. The negative control was star polymers without siRNA, as this provided a point of comparison to the star polymer-siRNA polyplex. The star polymer alone was expected to have a more positive zeta potential and a smaller diameter compared to the polyplex.

TABLE 4
Zeta potential and hydrodynamic volume measurement
after siRNA(F)^{a} complexation^{b}

| | | | Star Polymer | |
|---|---|---|---|---|
| | | | complexed with siRNA | |
| | Star Polymer only | | NSP-S_{SSX}-C/ | NSP-S_{SSX}-qC/ |
| | NSP-S_{SSX}-C | NSP-S_{SSX}-qC | siRNA(F) | siRNA(F) |
| ζ potential | 5.0 ± 0.3 | −1.2 ± 0.3 | −6.5 ± 1.2 | −4.6 ± 1.0 |
| (mV) | | | | |
| Dₕ (nm) | 7.7 ± 0.8 | 8.3 ± 0.7 | 12.6 ± 0.6 | 11.6 ± 0.3 |

^{a}siRNA(F); FITC-conjugated control siRNA
^{b}Both measurements were carried under DNase/RNase-free distilled water (Neutral pH)

### Results

The zeta potential varied from 5.0 to −6.5 mV (NSP-S_{SSX}-C) and from −1.2 to −4.6 mV (NSP-S_{SSX}-qC) after siRNA(F) complexation, respectively. Furthermore, the hydrodynamic volume of the siRNA complexed PEG star polymers was slightly changed due to the electrostatic interaction between the cationic star core and anionic siRNA, indicating successful complexation.

### Gel Retardation Assay

The siRNA to polymer complexation ratio was determined using a gel retardation assay. Determination of this ratio is crucial for determining the dosing capabilities of experimental polymers in subsequent studies. Gel retardation assays take advantage of a sample's size and charge to determine the extent of polyplex interaction. Ethidium bromide is a DNA intercalator that inserts itself into the spaces between the base pairs of the double helix. Since the siRNA used in this assay is double-stranded, the ethidium bromide acts as a fluorescent dye for free siRNA when viewed under UV light.

In this assay, 20 μL samples of varying polymer:siRNA ratio (ng:ng) were loaded into a 2% agarose gel and ran for 30 minutes at 100 V. Ethidium bromide was incorporated into the gel and the running buffer at a concentration at 0.5 μg/μL as a means for visualizing the siRNA within the gel. While then gel was run, uncomplexed siRNA from each sample migrated towards the cathode because of its anionic nature. When the siRNA in a sample was fully complexed with the star polymer, there was no uncomplexed siRNA to migrate down the gel and as a result, no siRNA band was visible on the gel. On the other hand, at ratios where uncomplexed siRNA were present siRNA bands were visible and full complexation had not been achieved. Photomicrographs were taken of the gels under UV transillumination.

### Results

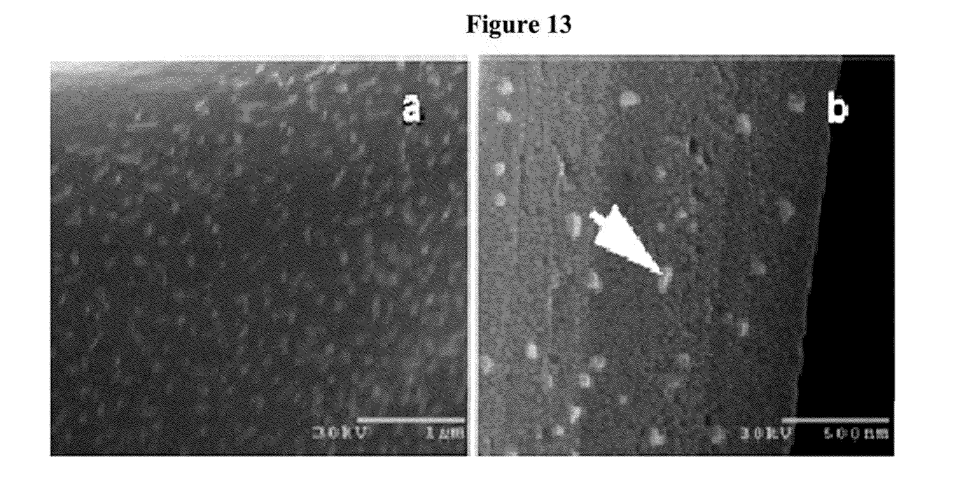

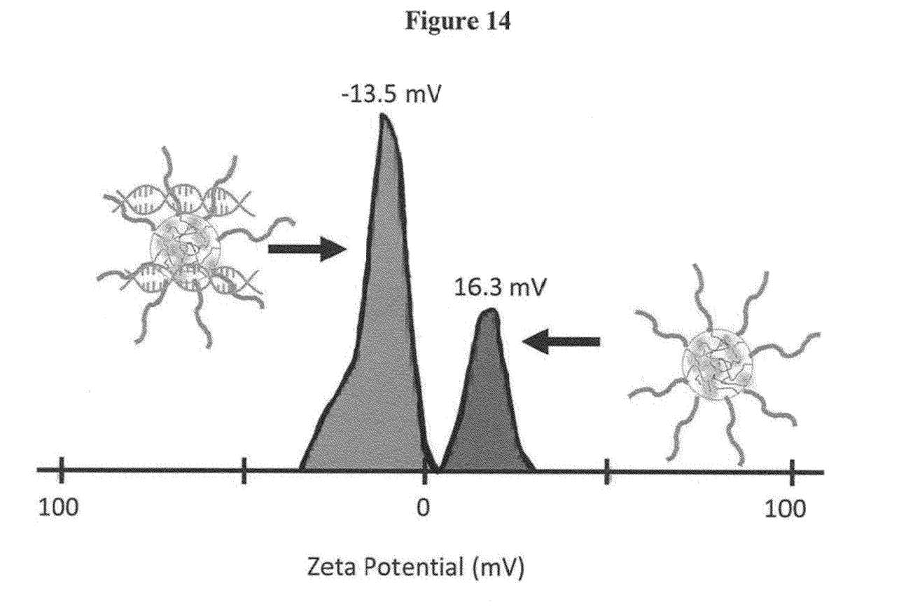

Gel retardation assay for NSP-S_{SSX}-N with a complexation time of 4 hours. Faint siRNA bands were still visible at 8000:1 and 10000:1 ratios, indicating that full complexation had not yet been reached. At a ratio of 15000:1, no siRNA band was detectable. This suggested that the requisite complexation ratio for a 4-hour complexation with a neutral star polymer is 15000:1. The gel retardation assay results for NSP-S_{SSX}-C with a complexation time of 4 hours, even at a ratio of 15000:1, suggested a faint siRNA band, suggesting that higher ratios were required to achieve full complexation within 4 hours. However, gel retardation assay results for NSP-S_{SSX}-qC with a complexation time of 4 hours displayed no siRNA band at the expected distance for any ratio above 1000:1 indicating that full complexation was achieved within 4 hours at a lower polymer:siRNA ratio than NSP-S_{SSX}-N or NSP-S_{SSX}-C. For samples with siRNA to nano-structured polymers (FIG. 13) at ratios of 8,000 and 10,000 of NSP-S_{SSX}-N to siRNA (ng:ng), a faint siRNA band was visible indicating that full complexation had not been achieved. However, at a ratio of 15,000, no siRNA band was visible, suggesting full complexation. In the case of NSP-S_{SSX}-C (FIG. 14), even at a ratio of 15,000, a faint siRNA band was still visible. This suggested that either higher ratios of NSP-S_{SSX}-C to siRNA are required to achieve full complexation, or more complexation time is required.

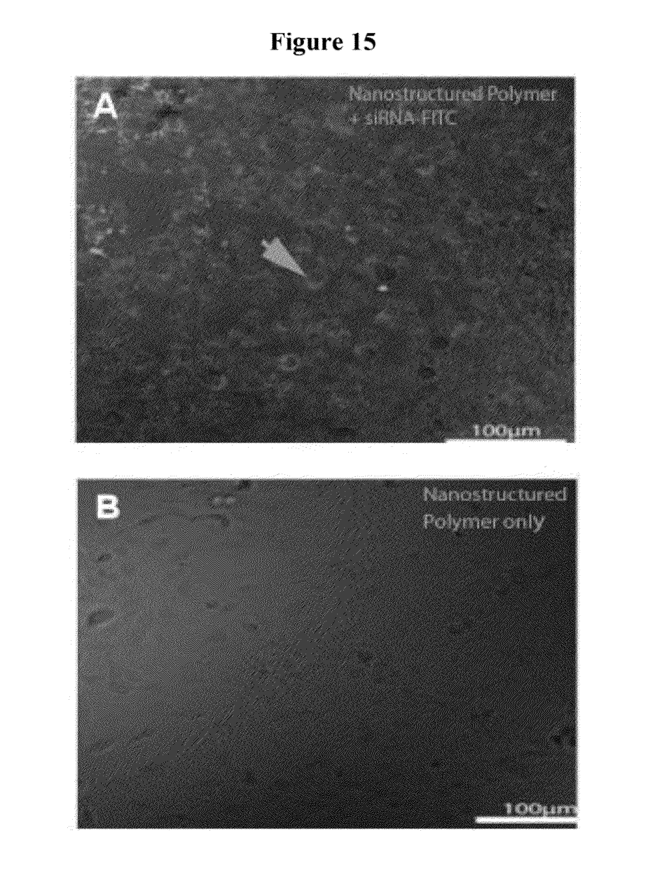

In the case of NSP-S_{SSX}-qC (FIG. 15), the lack of visible siRNA bands at ratios greater than 1000 indicated that complete siRNA complexation with the star polymer had occurred. As such, it is evident from these studies that the NSP-S_{SSX}-qC star polymer had the most efficient siRNA complexation properties among the three types of star polymers tested.

Therefore in one embodiment, star polymers with cationic cores and PEO arms have low cytotoxicity and can complex with negatively charged siRNA via electrostatic interaction. The nano-structured star polymers deliver model siRNA's into the cells (FIG. 13). In this embodiment an exemplary cationic PEO-based star polymer is synthesized, cytotoxicity and siRNA complexation is determined and murine myoblast cells (C2C12) are transfected. The use of PEO-based cationic star polymer resulted in 85-90% MC3T3 cell viability after 48 hours. Zeta potential analysis reveals robust complexation of siRNA's with star nano-structured polymer (FIG. 14). The negatively charged siRNA binds to the core of the positively charged nano-structured star polymers. In vitro transfection studies after 48 hrs show robust siRNA transfection into C2C12 cells (FIG. 15A). In contrast, the control groups that did not receive any FIDC conjugated siRNA (FIG. 15B) do not show any fluorescence. Thereby demonstrating that compositionally tailored cationic star polymers with PEO arms are biocompatible and have robust siRNA complexation capacity, showing that AC star nano-structured polymers can be effectively used for siRNA complexation and delivery.

### 1 E) Biocompatibility of NSP-S by Live/Dead Cell Staining

Live/Dead staining is a qualitative cytotoxicity assay that is commonly used to determine cell viability. The assay consists of using two reagents: the acetomethoxy derivative of calcein (calcein AM) and ethidium homodimer-1. Once calcein AM enters the cell, active intracellular esterases convert it to calcein resulting in a green fluorescence (excitation 495 nm, emission 515 nm). Ethidium homodimer-1 is only able to enter cells with damaged membranes and can intercalate with nucleic acid to increase its red fluorescence signature (excitation 495 nm, emission 635 nm). Through observing samples exposed to NSPs using fluorescent microscopy, a qualitative assessment of NSP biocompatibility can be determined.

Human mesenchymal stem cells (hMSCs) and murine calvarial pre-osteoblasts (MC3T3-E1.4) were seeded at a density of 100,000 cells/mL in 6 well tissue culture plates. Star polymers were added to the cells 48 hours later and cultured for an additional 48 hours. Prior to preparation for live/dead staining, the positive control wells were exposed to 70% ethanol for 1 hour. Cells were then washed with 1×PBS before being exposed to calcein AM and ethidium homodimer-1. For the ethidium homodimer-1, 20 μL of the supplied 2 mM solution was diluted in 10 mL of 1×PBS to produce a 4 μM EthD-1 solution. Next, 5 μL of the provided 4 mM calcein AM solution was added to the 4 μM EthD-1 solution. The cells were incubated with the dye solution for 30 minutes and rinsed twice with 1×PBS to remove any residual dye. Cells were then imaged by a fluorescent microscope using a FITC filter for calcein and Rhodamine filter for EthD-1. Images were taken a magnification of 10×.

The objective of this assay was to determine the biocompatibility of the NSP-S. Cells alone were used as a negative control and cells treated with ethanol were the positive control. The experimental groups were the three star polymers: NSP-S-SSX-0% N, NSP-S-SSX-5% N, NSP-S-SSX-5% QN at a concentration of 800 μg/mL.

### Results

From a qualitative standpoint, both hMSCs and MC3T3s exposed to NSP-S displayed similar cell viability compared to the negative control. The ethanol had an immediate effect on cell viability, as seen with the amount of red fluorescence under rhodamine filter, which indicates cell death. A dim green fluorescence can be observed for the positive control. This suggests that small amounts of active esterases are still present even after ethanol treatment. However, the distribution of green fluorescence and lack of distinct cells suggests that the damaged plasma membrane also allowed esterases to escape, thus causing a bloom in green fluorescence. From these images, it can be concluded that the NSP-S are biocompatible. This result supports the cytotoxicity data from the MTS and LDH assays reported above.

### Example 2

### Synthesis of Star Polymers with Expanded Cationic Cores

N-EGDMA (PEG-poly(DMAEMA-co-EGDMA) star polymers with different amounts of DMAEMA (i.e., cationic motifs) and EGDMA (i.e., crosslinker) were prepared via ATRP and are summarized in Table 5 below. Initial N-EGDMA star polymers contained 0.5 mmol of DMAEMA (84.2 μL) (HC2-79-A, previously defined as 5 wt % DMAEMA with EGDMA and used for the control experiment of degradation study) were prepared using the following formulation of PEGMA (M_{w}=2,080; 2.08 g, 1 mmol), EGDMA (188.6 μL, 1 mmol), CuBr (27.3 mg, 0.19 mmol), CuBr₂ (2.2 mg, 0.01 mmol), HMTETA (54.4 μL, 0.2 mmol), and EBiB (29.6 μL, 0.2 mmol) in 10 ml MeOH (0.5 ml Toluene) at 60° C. Conversion of DMAEMA and EGDMA, in these initial stars, was determined by gas chromatography (GC) to have values of 92% and 98%, respectively. N-EGDMA stars polymers were then synthesized with varying amounts of cationic motifs to include 1.0 mmol of DMAEMA (168.4 μL, HC2-78-B), 2.0 mol of DMAEMA (336.8 μL, HC2-78-C), and 4.0 mmol of DMAEMA (673.6 μL, HC2-78-D) with same procedure as HC2-78-A. Apparent molecular weights of HC2-78-A through D showed almost identical values (i.e. M_{w}˜30k) determined with THF GPC after dialysis purification by dialysis (MWCO 15k) for 4 days. The DMAEMA/EGDMA conversions of HC2-78-B, C, and D were 82%/90%, 81%/87%, and 77%/80%, respectively. In addition, the amount of nitrogen contained within the star polymers increased progressively from 23 to 151, from sample HC2-78-A to HC2-78-D. Therefore, stars polymers with variable amounts of nitrogen could be successful synthesized and later screened for their efficiency as vehicles for siRNA delivery. Similar experiments were then conducted instead with 2 eq. or 2.0 mmol of EGDMA (i.e., HC2-82-A though C) in all cases. This series is similar to the last series except with a larger fraction of EGDMA crosslinker aimed to provide a larger core structure. Samples contained 1.0, 2.0, and 4.0 mmol of DMAEMA for samples HC2-82-A, HC2-82-B, and HC2-82-C respectively. On average slightly larger M_{w,MALLS} values were observed and amounts of nitrogen were accomplished with larger loadings of DMAEMA. Synthesis of the most expanded core star polymers, designated as HC2-83-A and B, were prepared with 2.0 mmol of DMAEMA/4.0 mmol of EGDMA and 4.0 mmol of DMAEMA/4.0 mmol of EGDMA, respectively. After 24 hours the reaction products obtained were macroscopic gels instead of star polymers, which was due to the excess amount of EGDMA.

TABLE 5
PEG-Based Star Polymers with Expanded Core (N-EGDMA, nitrogen with
EGDMA cross-linker)

| | | | | | | | Nitrogen |
|---|---|---|---|---|---|---|---|
| | M_{w, THF GPC} | | | | M_{w,MALLS} | | atoms per |
| sample | (×10³) | M_{w}/Mₙ | conv,_{DMAEMA} | conv, _{EGDMA} | (×10³) | # of arms | star |
| HC2-78-A^{a} | 31.2 | 1.32 | 92% | 98% | 115.0 | 49 | 23 |
| HC2-78-B^{b} | 31.0 | 1.33 | 82% | 90% | 114.8 | 48 | 40 |
| HC2-78-C^{c} | 31.2 | 1.33 | 81% | 87% | 120.8 | 48 | 79 |
| HC2-78-D^{d} | 29.8 | 1.34 | 77% | 80% | 120.8 | 43 | 151 |
| HC2-82-A^{e} | 32.9 | 1.33 | 90% | 96% | 135.3 | 52 | 47 |
| HC2-82-B^{f} | 30.2 | 1.35 | 87% | 90% | 124.7 | 46 | 80 |
| HC2-82-C^{g} | 29.9 | 1.35 | 82% | 88% | 135.4 | 46 | 151 |

| | |
|---|---|
| HC2-83-A^{h} | Not available - Gelled Reaction |
| HC2-83-Bⁱ | Not available - Gelled Reaction |

All reactions were conducted in 10 ml methanol and 0.5 ml toluene for 24 hours at 60° C.
^{a}[PEGMA]₀/[DMAEMA]₀/[EGDMA]₀/[EBiB]₀/[CuBr]₀/[CuBr₂]₀/[HMTETA]₀ = 1/0.5/1/0.2/0.19/0.01/0.2 (PEG)₄₉-poly(DMAEMA₂₃-co-EGDMA₄₈).
^{b}[PEGMA]₀/[DMAEMA]₀/[EGDMA]₀/[EBiB]₀/[CuBr]₀/[CuBr₂]₀/[HMTETA]₀ = 1/1.0/1/0.2/0.19/0.01/0.2 (PEG)₄₈-poly(DMAEMA₄₀-co-EGDMA₄₄).
^{c}[PEGMA]₀/[DMAEMA]₀/[EGDMA]₀/[EBiB]₀/[CuBr]₀/[CuBr₂]₀/[HMTETA]₀ = 1/2.0/1/0.2/0.19/0.01/0.2 (PEG)₄₈-poly(DMAEMA₇₉-co-EGDMA₄₃).
^{d}[PEGMA]₀/[DMAEMA]₀/[EGDMA]₀/[EBiB]₀/[CuBr]₀/[CuBr₂]₀/[HMTETA]₀ = 1/4.0/1/0.2/0.19/0.01/0.2 (PEG)₄₃-poly(DMAEMA₁₅₁-co-EGDMA₃₉).
^{e}[PEGMA]₀/[DMAEMA]₀/[EGDMA]₀/[EBiB]₀/[CuBr]₀/[CuBr₂]₀/[HMTETA]₀ = 1/1.0/2/0.2/0.19/0.01/0.2 (PEG)₅₂-poly(DMAEMA₄₇-co-EGDMA₁₀₀).
^{f}[PEGMA]₀/[DMAEMA]₀/[EGDMA]₀/[EBiB]₀/[CuBr]₀/[CuBr₂]₀/[HMTETA]₀ = 1/2.0/2/0.2/0.19/0.01/0.2 (PEG)₄₆-poly(DMAEMA₈₀-co-EGDMA₈₃).
^{g}[PEGMA]₀/[DMAEMA]₀/[EGDMA]₀/[EBiB]₀/[CuBr]₀/[CuBr₂]₀/[HMTETA]₀ = 1/4.0/2/0.2/0.19/0.01/0.2 (PEG)₄₆-poly(DMAEMA₁₅₁-co-EGDMA₈₁).
^{h}[PEGMA]₀/[DMAEMA]₀/[EGDMA]₀/[EBiB]₀/[CuBr]₀/[CuBr₂]₀/[HMTETA]₀ = 1/2.0/4/0.2/0.19/0.01/0.2
ⁱ[PEGMA]₀/[DMAEMA]₀/[EGDMA]₀/[EBiB]₀/[CuBr]₀/[CuBr₂]₀/[HMTETA]₀ = 1/4.0/4/0.2/0.19/0.01/0.2

TABLE 6
Percentage of siRNA complex with STAR polymers
under different weight ratio

| | 500:1 | 1000:1 | 2000:1 | 4000:1 |
|---|---|---|---|---|
| HC2-78-A | 0.6% | 30% | 28% | 32% |
| HC2-78-B | 23% | 42% | 56% | 61% |
| HC2-78-C | 56% | 57% | 86% | 88% |
| HC2-78-D | 60% | 83% | 89% | 91% |
| HC2-82-A | 12% | 18% | 60% | 85% |
| HC2-82-B | 25% | 35% | 63% | 84% |
| HC2-82-C | 76% | 87% | 86% | 87% |

A trend from this series of experiments is that increasing the number of monomer units comprising a complex forming functionality (e.g., nitrogen) provides more efficient complexation, especially at low siRNA/STAR ratio. Higher cross-linking density, (e.g. column: 500:1, rows: HC2-78-B vs. HC2-82-A) generally reduces the extent of the complexation (23% vs. 12%). However, at an increasing siRNA/STAR weight ratios this trend is less pronounced and may be reversed at the highest N content (e.g., column: 500:1, rows: HC2-78-D vs. HC2-82-C).

### Example 3

### Synthesis of Short-Arm PEG (DP=22) Stars with Cationic Core

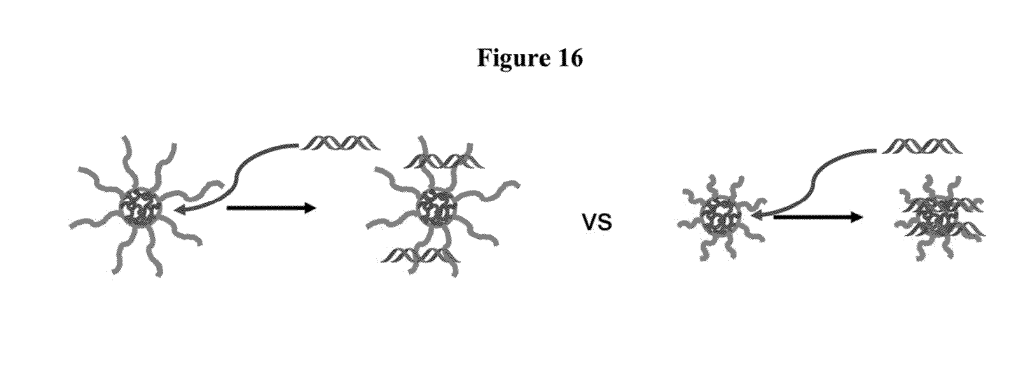

FIG. 16 illustrates the synthesis of short-arm PEG (DP=22) stars with cationic core, which results in reduced congestion in stars with lower molecular weight. As shown in FIG. 16, siRNA accessibility to the cationic core is another parameter of efficient delivery of the siRNA cargo to the targeted cells. As model study, HC2-84-A to C were prepared (results are shown in Table 7) and under purification. All reactions were stopped after 24 hours. Conversion of DMAEMA and EGDMA was calculated by GC. Another approach to reduced congestion is expansion of the core by increasing fraction of incorporated mono-vinyl monomer, optimally conducted at low concentration of reagents in reaction medium.

TABLE 7
Short PEG Arm Star Polymers with Cationic Core.

| | M_{w,THFGPC} | | | | M_{w, MALLS} | | |
|---|---|---|---|---|---|---|---|
| sample | (×10³) | M_{w}/Mₙ | conv,_{DMAEMA} | conv,_{EGDMA} | (×10³) | # of arms | N/star |
| HC2-84-A^{a} | 24.0 | 1.22 | 95% | 94% | N/A | N/A | N/A |
| HC2-84-B^{b} | 25.1 | 1.31 | 92% | 92% | N/A | N/A | N/A |
| HC2-84-C^{c} | 25.1 | 1.30 | 89% | 88% | N/A | N/A | N/A |

All reactions were conducted in 10 ml methanol and 0.5 ml toluene for 24 hours at 60° C.
^{a}[PEGMA]₀/[DMAEMA]₀/[EGDM]₀/[EBiB]₀/[CuBr]₀/[CuBr₂]₀/[HMTETA]₀ = 1/0.3/0.3/0.2/0.19/0.01/0.2
^{b}[PEGMA]₀/[DMAEMA]₀/[EGDM]₀/[EBiB]₀/[CuBr]₀/[CuBr₂]₀/[HMTETA]₀ = 1/0.6/0.3/0.2/0.19/0.01/0.2
^{c}[PEGMA]₀/[DMAEMA]₀/[EGDM]₀/[EBiB]₀/[CuBr]₀/[CuBr₂]₀/[HMTETA]₀ = 1/0.9/0.3/0.2/0.19/0.01/0.2

### Example 4

### Quantification of Complexation of NSP-S and siRNA

### Quantification by Ribogreen Assay

One strategy for improvement was to synthesize star polymers with variable core sizes by changing the relative amount of crosslinker, and a second strategy was to produce cores which contained a larger fraction of cationic motifs. Each of these star delivery systems were synthesized via atomic transfer radical polymerization (ATRP) using the previously described PEG macro-monomer. It was anticipated that PEG star polymers with expanded cores will be beneficial for siRNA complexation by improving accessibility and larger concentrations of cationic charges will enable improved loading capacities of siRNA to each star polymer.

### Ribogreen Assay

In order to quantify the complexation ratio between siRNA and cationic star polymers, a RNA quantitation reagent, RiboGreen® (Invitrogen, Carlsbad, Calif.) was selected. RiboGreen® RNA quantitation reagent is a sensitive fluorescent nucleic acid stain for quantifying RNA in solution as little as 1 ng/mL RNA with a fluorescence microplate reader. The excitation maximum for RiboGreen reagent bound to RNA is ˜500 nm and the emission maximum is ˜525 nm. This sensitivity exceeds that achieved with ethidium bromide-based assays (used in gel retardation assay) by 200-fold and exceeds that achieved with ultraviolet absorbance determination (used in absorbance at 260 nm) by 1000-fold.

siRNA was dissolved in RNase-free water, specific amounts of cationic star polymers were added. The weight ratio ranged from 500:1 to 4,000:1 (polymer/siRNA, ng:ng). After 3 hours complexation in room temperature, we precipitated the uncomplexed siRNA in 80% ethanol, 20 mM ammonium acetate and 400 μg/ml glycogen. The precipitated siRNA was pelleted by centrifuge. Supernatants were removed and pellets were air dried to remove the residual ethanol. After being dissolved in RNase-free water, 100 μl aliquot were mixed with 1:500 diluted Ribogreen® reagents. Fluorescence intensity was read via TECAN® spectrophotometer. The NSP-S-SSX-5% QN sample, which is the cationic star polymer containing a quaternized DMAEMA core, complexed the most siRNA among the three types of NSP-S. This is demonstrated by the relatively high percentage of siRNA complexed under a certain level of polymer weight ratio.

### Quantification of the Complexation by Zeta Potential Analysis

NSP-S/siRNA complexes were prepared at various nitrogen/phosphate molar ratios from 0.2:1 to 10:1 by adding various amounts of siRNA with 50 μM concentration. Complexation was carried at 4° C. for 2 h and then incubated for 30 min at room temperature. Zeta potential and size distribution of each sample were determined using three repeats (i.e., a trio), each trio was in turn measured three times using a Zetasizer Nano (Malvern Instruments, UK). Solution 1 mg/mL of star polymer under DNase/RNase free water was prepared. Aliquots of siRNA solution were added to the star polymer solution, corresponding to molar N/P ratios based on the overall star composition and siRNA. NSP-S-SSX-5% N, NSP-S-SSX-5% QN, and NSP-S-SSX-0% N were evaluated for complexation to siRNA by zeta potential measurements.

Incorporation of cationic monomers DMAEMA or QDMAEMA to the star polymers facilitated electrostatic complexation with the negatively charged siRNA. The initial zeta potential of NSP-S-SSX-5% N and NSP-S-SSX-5% QN was 22.5±1.32 mV and 2.38±0.73 mV while the zeta potential of NSP-S-SSX-0% N was −6.68±1.90 mV because of different core charges. The zeta potential value of NSP-S-SSX-5% N decreased from 22.9±1.78 mV to −26.3±1.27 mV with increasing siRNA amount (with degreasing N/P ratio from 10 to 0.2). In the case of NSP-S-SSX-5% QN, the variation of zeta potential on N/P ratio is small, indicating the efficacy of siRNA complexation of NSP-S-SSX-5% N is greater than for NSP-S-SSX-5% QN. The hydrodynamic volume of the NSP-S complexed with siRNA did not change significantly with N/P ratios (for example, hydrodynamic diameter of NSP-S-SSX-5% QN varied from 12.53±1.00 to 12.82±1.00 nm for N/P=0.2 and 10, respectively), suggesting siRNA complexed star polymers stayed as individual polymers without aggregation.

TABLE 8
Percentage of siRNA complex incorporated into
star polymers under different ratios

| | 500:1 | 1000:1 | 2000:1 | 4000:1 |
|---|---|---|---|---|
| HC2-78-A | 0.6% | 30% | 28% | 32% |
| HC2-78-B | 23% | 42% | 56% | 61% |
| HC2-78-C | 56% | 57% | 86% | 88% |
| HC2-78-D | 60% | 83% | 89% | 91% |
| HC2-82-A | 12% | 18% | 60% | 85% |
| HC2-82-B | 25% | 35% | 63% | 84% |
| HC2-82-C | 76% | 87% | 86% | 87% |

Characterization of the siRNA Release Kinetics with NSP-S

Based on the successful complexation and degradation studies for cationic star polymers the siRNA release kinetics were measured during degradation. siRNA and cationic star polymers were initially complexed and the complex was treated with 10 mM GSH under nitrogen gas up to 9 days. The change in concentration of the uncomplexed siRNA in the solution as well as the total siRNA level was measured. The change of the percentage of uncomplexed siRNA to the total siRNA level yielded the siRNA releasing kinetics data profile. 100 ng/ml siRNA was complexed with 0.2 mg/ml cationic star polymers at room temperature for 3 hours. Freshly prepared GSH solution was added to get the final concentration of 10 mM. After mixing, the solution was bubbled with pure nitrogen gas for 1 hour to remove the solubilized oxygen in order to keep GSH active for long time. The tubes were filled with nitrogen gas then capped and sealed to ensure airtight. At each temporal point, 200 μl solutions were transferred for siRNA precipitation and Ribogreen quantification assay.

Our preliminary study demonstrated that 500 mM ammonium acetate precipitated total siRNA, both complexed and uncomplexed due to the ion strength that dissociated the static interactions of complexation, whereas low ion strength such as 20 mM ammonium acetate is sufficient to precipitate free siRNA in the solution but not the complexed siRNA. siRNA was released into the solution as the uncomplexed form in a time-dependent manner. There was no siRNA release in the control group over 9 days, that is, in the absence of 10 mM glutathione. At day 9, almost 100% siRNA was present in the solution as the uncomplexed form, which confirmed 100% release.

Demonstration of siRNA Internalization Via Cationic Nano-Structured Polymers

Since siRNA's are electrostatically repulsed by the plasma membrane, this necessitates a delivery vehicle to intracellularly transport siRNA. The rationale of cellular internalization experiments is to determine the efficacy of the nano-structured polymers (NSP) in delivering siRNA into the cytoplasm. For these experiments, fluorescent negative control siRNA (siRNA conjugated with the Cy3 fluorophore) were complexed with non-fluorescent NSP's and then introduced to cell culture. The stains commonly used to identify cells are 4′,6-diamidino-2-phenylindole (DAPI), which intercalates with DNA in the nuclei, and Phalloidin, which binds to actin filaments that comprise the cytoskeleton.

Human mesenchymal stem cells (hMSCs) were seeded at a density of 150,000 cells/well on sterile glass coverslips placed in 6-well plates. Cells were incubated for 24 hrs before receiving a media change. NSP and siRNA were allowed to complex at 4° C. for 1 hr. NSP, Cy3-siRNA, and polyplex solutions were subsequently added to cells, and then incubated for another 24 hrs. The cells were fixed using 4% formaldehyde in PBS solution, and washed in 1×PBS three times for 10 minutes each. During the second wash, 1 μL of Triton-X100 was added to each well. Each coverslip was then removed from the 6 well plate, and covered in 200 μL PBS solution containing 0.5% DAPI stain and 0.5% Phalloidin stain for 90 min at room temperature. The coverslips were washed again with 1×PBS for 10 minutes, and imaged using confocal microscopy at magnifications of 40× (cells alone only) and 20× (all other samples).

An examination of the images sowed that an increase in Cy3 fluorescence was detected only in the polyplex treatment cohorts. None of the controls (i.e., cells alone, cells with naked siRNA, and cells with NSP alone) exhibited Cy3 fluorescent, thus indicating NSP was necessary and capable of siRNA internalization. This profound accomplishment is compelling and underscores a key property of our technology.

These examples unequivocally demonstrate that the star polymers are biocompatible, although they appear to have a slight inhibitory effect on cell proliferation.

### Example 5

### Synthesis and Characterization of Core-Shell Nanogels

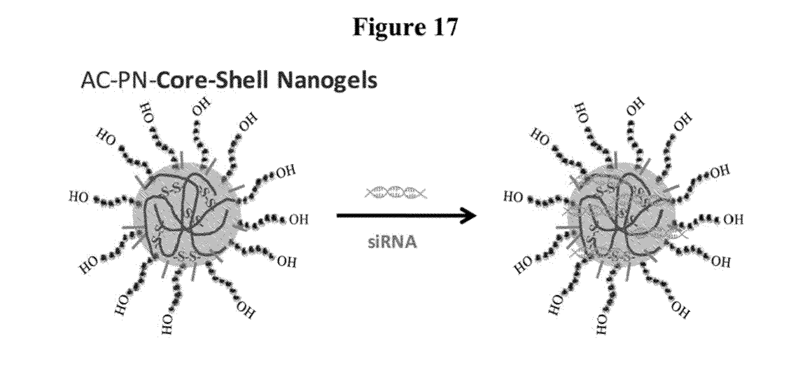

Core-shell nanogels consist of a core-shell structure as depicted in FIG. 17. The peripheral shell is composed of a water soluble biocompatible polymer, exemplified herein by PEO, which can be equipped with functional groups for subsequent scaffold attachment.

The core structure contains degradable linkages (i.e., disulfides) and cationic motifs enabling efficient siRNA complexation similar to those used in the previously presented star polymers. Advantages of the core-shell AC-PNs (NSP-NG_{SSX}-N) include improved siRNA capacity in comparison to star AC-PNs (NSP-S_{SSX}-N) and enhanced cellular internalization rates in comparison to nanogel AC-PNs (NSP-CS_{SSX}-N). In addition, core-shell AC-PNs may provide rapid dissolution in aqueous media without aggregation problems observed in some previously synthesized nanogel systems.

Synthesis of core-shell AC-PNs involved two main steps: (1) creation of a reactive surfactant block copolymer and (2) employment of the reactive surfactant in a miniemulsion polymerization. The first step was accomplished by using atom transfer radical polymerization (ATRP) to produce a block copolymer of predetermined molecular weight (Mₙ) with a low molecular weight distribution (M_{w}/Mₙ). This block copolymer is composed of hydrophilic and biocompatible oligo(ethylene oxide) methacrylate (OEOMA₄₇₅) blocks and a hydrophobic 2-methoxyethyl methacrylate (MEMA) block. An amphiphilic block copolymer is required to promote self-assembling behavior in the subsequent miniemulsion step. The block copolymers were precisely synthesized with pre-determined molecular weights indicated by the strong correlation existing between theoretical (M_{n,theo}) and experimental (M_{n,gpc}) molecular weight values. Furthermore, M_{w}/Mₙ values in all cases were maintained below 1.3, with exceptionally narrow values obtained by using sequential monomer addition. Specific reaction conditions for each polymerization are supplied in the footnotes within Table 9.

TABLE 9
Resulting P(OEOMA₄₇₅)-b-P(MEMA) block copolymers synthesized via ATRP.

| | P(OEOMA₄₇₅) | | | | | P(OEOMA₄₇₅)-b-P(MEMA) | | | | |
|---|---|---|---|---|---|---|---|---|---|---|
| Ent. | time | p_{NMR} | | | | time | p_{NMR} | | | |
| (#) | (h) | (%) | M_{n,theo} | M_{n,GPC} | M_{w}/Mₙ | (h) | (%) | M_{n,theo} | M_{n,GPC} | M_{w}/Mₙ |
| l^{a} | — | — | — | 12,000 | 1.15 | 15 | 72 | 31,859 | 32,700 | 1.25 |
| 2^{b} | 4.66 | 76 | 9,268 | 8,700 | 1.16 | 2.0 | 36 | 11,341 | 12,600 | 1.15 |
| 3^{c} | 3.2 | 90 | 21,637 | 19,500 | 1.12 | 6.17 | 53 | 36,914 | 34,300 | 1.16 |

^{a}[MEOMA]/[MI]/[dNbpy]/[Cu^{II}Cl₂]/[CuCl₂] = 200/1/4/0.02/0.18; [MEMA] = 1.5 in anisole; Temp. = 50° C.; utilized macroinitiator (MI) = 12,000 g/mol.
^{c}[OEOMA₄₇₅]/[EBPA]/[TPMA]/[Cu^{II}Br₂]/[Sn^{II}(EH)₂] = 25/1/0.134/0.045/0.45; [MEMA]/[EBPA] = 25/1 [OEOMA₄₇₅] = 0.34 M (85 (v/v) % anisole), Temp. = 60° C.
^{d}[OEOMA₄₇₅]/[EBPA]/[TPMA]/[Cu^{II}Br₂]/[Sn^{II}(EH)₂] = 50/1/0.27/0.09/0.9; [MEMA]/[EBPA] = 200/1 [OEOMA₄₇₅] = 0.34 M (85 (v/v) % anisole), Temp. = 60° C.

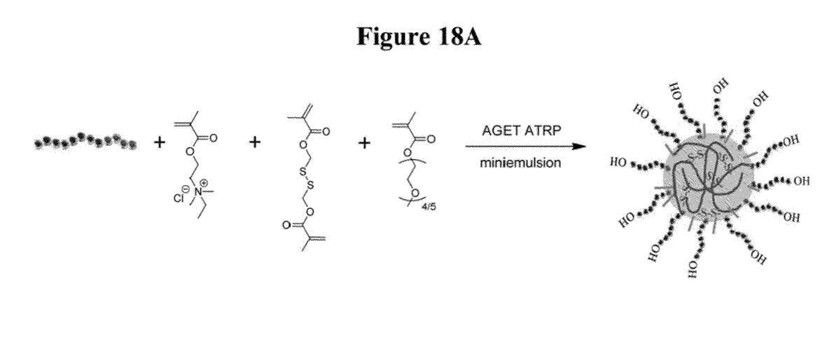

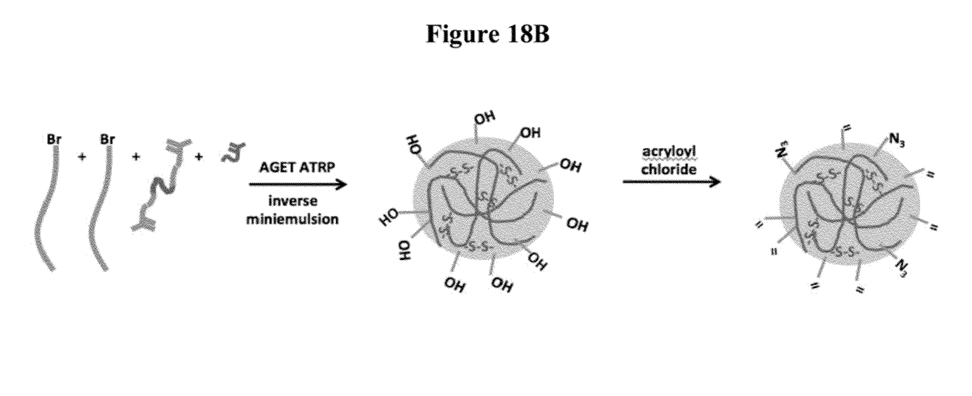

As stated earlier, synthesis of core-shell AC-PNs involves two steps, the second of which is the employment of the reactive surfactant (i.e. P(OEOMA₄₇₅)-b-P(MEOMA)) in a mini-emulsion polymerization as shown in FIG. 18A. The mini-emulsion enables formation of the core-shell structure while also incorporating both the degradable and cationic motifs into the core of the AC-PN.

Self-assembling behavior is a critical parameter for a successful mini-emulsion and was evident in DLS of the structures formed by self assembly, particles with a size of 34 nm. Structures of this size indicate that the block copolymers are capable of assembling into micelles. Three acrylated cationic polymeric nanomaterials (AC-PNs) were synthesized; a typical nanogel structure is depicted in FIG. 18B.

Nanogels consist of ca. 200 nm sized gels were prepared via Activator Generated by Electron Transfer (AGET)—Atom Transfer Radical Polymerization (ATRP) through an inverse-mini-emulsion (water-in-oil) process. In order to complex siRNAs with nanogels, the nanogels are prepared with a predetermined weight fraction of cationic monomer. The nanogels are synthesized with a degradable cross-linker (disulfide) to release complexed siRNA and allow degradation of the nanogel structure into polymer fragments which are capable of excretion via renal filtration.

Culture experiments using Human Umbilical Vein Endothelial Cells (HUVEC) and the above nano-structured copolymers were undertaken to document any morphological changes and growth inhibition due to the polymer treatment. In addition, quantitative PCR primers were designed to assess the levels of the certain human genes associated with either apoptosis or oxidative stress. The genes identified in Table 1 were monitored to optimize siRNA-specific effects and differentiate between siRNA-specific and polymer-specific perturbations of gene expression.

Primer sets for these genes have been generated to be specific for mRNA by spanning large introns. Initial experiments actively validated these primer sets and Q-PCR arrays for both apoptosis and oxidative stress (SABioscience-QIAGEN) were conducted to standardize and validate the results from designed Q-PCR primer sets.

### Example 6

### Nanogels for siRNA Delivery

ATRP in inverse miniemulsion was used to prepare cationic nanogels with rationally selected properties. Nanogels consist of ca. 200 nm-sized gels which were prepared via Activators Generated by Electron Transfer (AGET) Atom Transfer Radical Polymerization (ATRP) through an inverse-mini-emulsion (water-in-oil) process. The monomers, crosslinker, catalyst and initiator were dissolved in the water phase and then dispersed with the aid of a surfactant into an oil phase. With the addition of a reducing agent the polymerization was initiated, and the water phase was crosslinked to yield latexes of ˜200 nm in size. The nanogels were designed with redox or acid sensitive cross linkers so that the materials can effectively release siRNA under appropriate biological signals. Four nanogels were prepared to determine the effect of cationic charge, crosslinking density on siRNA complexation (at a constant weight percent of q-DEMEMA) and effect of crosslinker on degradability.

Nanogel materials were synthesized and analyzed by zeta potential measurements to optimize siRNA complexation. To determine complexation, two nanogels were prepared with either a neutral (i.e., no cationic monomers were incorporated, Sample 36, NSP-NG-SSX-0% N) and or cationic core (i.e., containing w/w ˜5%, Sample 37, NSP-NS-SSX-5% QN). DLS and zeta data for NSP-NG-SSX-5% QN and NSP-NG-SSX-0% N show volume distribution of nanogels were monomodal and had low polydispersity (Sample 36: radius 183.6 nm, PDI—0.25; Sample—37: radius—170 nm, PDI—0.164) indicating that these materials are stable and were successfully prepared. The Zeta potential measurements of NSP-NG-SSX-5% QN revealed a 17.9 mV+/−0.902 value which would be expected from a 5% (w/w) loading of cationic monomer and, therefore, deemed suitable for use. Furthermore, NSP-NG-SSX-0% N had a zeta potential of −8.25 mV+/−0.554. This slightly negative value indicates a slightly negative charge exists on the neutral star polymers. This material was also deemed acceptable for siRNA complexation studies.

To determine the effect of crosslinking density, two nanogels were prepared and evaluated (Sample 37 (previous described) and Sample 50: radius—186 nm, PDI—low (FIG. 6A). The amount of crosslinker per monomer unit for Sample 50 was 1:42, whereas this value was decreased to 1:80 for Sample 37. The zeta potential of Sample 50 was 40 mV+/−4.31 while Sample 37 was 17.9 mV+/−0.902. After the previously mentioned characterization results, cytotoxicity and siRNA complexation analysis was carried out.

To examine the effect of crosslinking agent on the degradability of the nanogels two nanogels with the same loading of q-DEMEMA were prepared while varying the type of crosslinker. Sample 37 employed a disulfide-based crosslinker, which can be degraded by intercellular reducing agents. Sample 51 was synthesized to contain a poly(glycolic acid) based crosslinking agent that had been previously shown to degrade under acidic conditions. Furthermore, 0.5% (w/w) of a rhodamine-derived monomer was incorporated to visualize cellular internalization of the nanogel materials. Sample 51 (FIG. 6A) was shown to be well defined by DLS with a diameter of 260 nm and a zeta potential value of 26 mV.

In conclusion, cationic nanogels were characterized by varying nanogel properties to determine suitability for the biological application of siRNA delivery. The material properties were varied to account for charge, crosslinking density and crosslinker degradability. Therefore, NSP-NG was successfully prepared as indicated by dynamic light scattering and zeta potential analysis.

### In Vitro Degradation and Biocompatibility of Cationic Nanogels

Degradation studies were executed under physiological conditions to the degradation. A screen of nanogels with pHX and SSX crosslinked nanogels will be compared for cytotoxic effects and siRNA delivery capacity. In the literature it is noted that pH sensitive crosslinkers can more readily degraded. This can have implications in the rate of siRNA release and this will be tested. These studies were conducted under conditions similar to those detailed above and results demonstrated excellent biocompatibility between NSP-NG-SSX-5% N with MC3T3 cells. The NSP-NG-SSX-5% QN was cytotoxic in a dose-dependent manner. NSP-NG-SSX-5% QN is more positively charged (+40 mV) than NSP-NG-SSX-5% N (+20 mV), the increased cytotoxicity may be related to the increased positive charge.

MTS Assay was conducted as described above for the star complexes. The results indicated that the NSP-NGssx-N and NSP-NGssx-C nanogels were biocompatible with MC3T3-E1.4 cells. Furthermore, measurement of LDH-based cytotoxicity for MC3T3-E1.4 after 48 hrs exposure to nanogels demonstrated that the nanogel polymers did not induce elevated levels of LDH release. There was no statistical difference between the experimental cohort and the negative control.

In Vitro siRNA Complexation with Cationic Nanogels

The siRNA to NSP-NG ratio was determined using a gel retardation assay. Determination of this ratio is crucial for determining the dosing capabilities of polymers to delivery the siRNA into the cell. In this assay, 20 μL samples of varying NSP-NG:siRNA ratio (ng:ng) were loaded into a 2% agarose gel and run for 30 minutes at 100 V. Ethidium bromide was incorporated into the gel and the running buffer at a concentration at 0.5 μg/μL as a means for visualizing the siRNA within the gel. While the gel was run, uncomplexed siRNA from each sample migrated towards the cathode because of its anionic nature. When the siRNA in a sample was fully complexed with the NSP-NG, there was no uncomplexed siRNA to migrate down the gel and as a result, no siRNA banding was visible on the gel. On the other hand, at ratios where uncomplexed siRNA was present, siRNA bands were visible and full complexation had not been achieved. Photomicrographs were taken of the gels under UV transillumination. Cationic nanogels complexed more siRNA than the neutral type the more positively charged type, i.e., NSP-NG-SSX-5% QN demonstrated better complexation than the less positively charged, i.e., NSP-NG-SSX-5% N.

Ribogreen assay was also conducted. siRNA was dissolved in RNase-free water, specific amounts of NSP-NG were added to get a weight ratio from 200:1 to 1000:1 (NSP-NG/siRNA, ng:ng). After 3 hours complexation at room temperature, we precipitated the uncomplexed siRNA in 80% ethanol, 20 mM ammonium acetate and 400 μg/ml glycogen. The precipitated siRNA was pelleted by centrifuge. Supernatants were removed and pellets were air dried to remove the remaining ethanol. After dissolved in RNase-free water, 100 μl aliquot were mixed with 1:500 diluted Ribogreen® reagents. Fluorescence intensity was read via TECAN® spectrophotometer and indicated that the cationic nanogel polymers complexed more siRNA than the neutral type. The more positively charged type, i.e., NSP-NG-SSX-5% QN, and had better complexation than the less positively charged, i.e., NSP-NG-SSX-5% N.

In Vitro Cellular Internalization of siRNA Conjugated Cationic Nanogels

Compared to the cationic NSP-S, the cationic NSP-NG appears to provide a higher level of siRNA complexation. In order to validate whether cationic NSP-NG will be internalized into mammalian cells, they were conjugated with trimethyl rhodamine isothiocyanate (TRITC). TRITC is a fluorescent dye that has an excitation wavelength of 557 nm and emission wavelength of 576 nm. Its presence as a component of a cationic nanogel enables visualization inside cells by confocal microscopy. Confocal microscopy allows for imaging of a certain plane of an object by blocking out out-of-focus light using a spatial pinhole. The use of this technology allows one to distinguish between fluorescence within a cell from fluorescence on the surface of one.

MC3T3 cells were plated on glass cover slips at a concentration of 200,000 cells/mL in a 6-well tissue culture-treated polystyrene plate (400,000 cells per cover slip). At t=24 hours, cell medium was replaced with medium of three different compositions. These included cell culture medium with 200 μg/mL TRITC-nanogel, cell culture medium with 400 μg/mL TRITC-nanogel and regular cell culture medium (with no nanogels present). At t=48 hours, the cell medium was aspirated and cells were washed twice with PBS at 37 C. Cells were fixed in the dark with 4% formaldehyde for 15 minutes. After 3 washes with 1×PBS, each cover slip was placed feature side down on 200 μL droplets of staining solution. The staining solution consisted of 2 μL of DAPI stain, 2 μL of phalloidin stain and 196 μL of PBS. After 60 minutes in the staining solution, each cover slip was placed into fresh 1×PBS and taken for confocal imaging. At 200 μg/mL TRITC-nanogels significant rhodamine fluorescence (green) is visible in the cytoplasm surrounding the cell nuclei. This is a very strong indicator of nanogel internalization at 200 μg/mL. The fluorescence at 400 μg/mL TRITC-nanogels appeared more intense than at 200 μg/mL, indicating a higher degree of nanogel internalization.

### Demonstration of Biocompatibility for Cationic Nanogel Polymers

MTS Assay was conducted as described above for the star complexes. The results indicated that the NSP-NGssx-N and NSP-NGssx-C nanogels were biocompatible with MC3T3 cells. Furthermore, measurement of LDH-Based Cytotoxicity for MC3T3-E1.4 after 48 hrs Exposure to Nanogels demonstrated that the nanogel polymers did not induce elevated levels of LDH release. There was no statistical difference between the experimental cohort and the negative control.

As stated above, the scaffold plays a key role as a material component involved in successful preventative treatment of HO. The scaffold provides critical elements of function to the overall composite material (i.e., scaffold+nanomaterial) by providing: (1) mechanical integrity for facile surgical implantation, (2) sequestration, transport, and containment of embedded nano-structured materials, (3) biocompatibility, (4) degradation and absorption, and (5) controlled release of cargo material (i.e., siRNA) over the desired window of treatment.

### Example 7

### Incorporation of rhBMP-2 into Collagen Scaffolds

Synthes XCM™ (which will be referred to herein as XCM) is biological tissue matrix is a sterile non-cross-linked 3-D matrix derived from porcine dermis. It undergoes Kensey-Nash's proprietary Optrix™ process that removes antigenic components from biological material while maintaining the native collagen structure and key extracellular matrix molecules used in tissue reconstruction.

rhBMP-2 Incorporation into Collagen Scaffolds

First, the void volume for loading rhBMP-2 into XCM scaffolds was determined. XCM biological tissue matrix was purchased from Synthes Corp. The matrix sheet was tailored into 5 mm in diameter and 1 mm in thickness implants via 5 mm biopsy punch. The implant scaffolds were stored in 1×PBS, transferred into a centrifuge filter tube and after centrifugation at 13,000 rpm for 2 minutes at room temperature, the solution that collected on the bottom was measured for the volume. We collected 7.5 μl solution from XCM scaffold of size 5 mm in diameter. This experiment was repeated 3 times; the void volume for loading rh-BMP-2 into XCM scaffolds is 7.5 μl.

We loaded 7.5 μl rhBMP-2 solution with different concentrations to the dry XCM scaffolds. The total amounts for rhBMP-2 loaded were 0, 5, 10 and 15 μg/scaffold. The same amounts of rhBMP-2 were loaded to the A collagen scaffolds. After loading, the XCM scaffolds were frozen in −80° C. for 1 hour and lyophilized. The A collagen scaffolds were air dried in room temperature for 2 hours. All scaffolds with rhBMP-2 were stored in −80° C. before use. All scaffolds were prepared aseptically.

In Vitro Characterization of rhBMP-2 Release Kinetics from Collagen Scaffolds and Bioactivity Analysis In Vitro rhBMP-2 Release Kinetics from Collagen Scaffolds

The dose of rhBMP-2 added to the scaffolds was 2.5 μg. The release media was composed of pH 7.4 in 1×PBS supplemented with 1% Bovine Serum Albumin (BSA) and 1% antibiotic/antimycotic. The releasates were collected every day until day 10. The rhBMP-2 concentration was measured by Enzyme-linked Immunosorbent Assay (ELISA) according to the protocol of kit purchased from R&D™ Systems (Catalog number DBP200). The collagen scaffold (identified as A-collagen) released rhBMP-2 faster than the XCM scaffold. The rhBMP-2 was released in a linear pattern from both scaffolds during the 10 days incubation. By day 10, the A collagen scaffold released 2 fold more rhBMP-2 in a cumulative amount as compared to XCM.

The releasates containing rhBMP-2 were collected from the release kinetics study. According to the concentrations of rhBMP-2 determined by ELISA, releasates were diluted with culture medium to prepare rhBMP-2 at a concentration of 50 ng/ml. HMSC cells growing in such medium were differentiated for 21 days. Medium was replaced with freshly diluted releasates every 3 days. Bioactivity of rhBMP-2 releasates will be determined by the effects on proliferation, differentiation and mineralization. DNA concentration will be measured for proliferation, alkaline phosphatase (ALP) activity for differentiation and calcium content for mineralization respectively.

### Example 8

### Establishment of Animal Model for In Vivo Ectopic Ossification

The Synthes XCM™ and A collagen scaffolds with rhBMP-2 protein were implanted in the thigh muscle of C57BL/6J mouse (8 weeks old) for 2 and 4 weeks.

TABLE 10
Experimental design for in vivo ectopic ossification induced by rhBMP-2

| | |
|---|---|
| 2 weeks | 4 weeks |

| | Scaffold A | Scaffold X | Scaffold A | Scaffold X |
|---|---|---|---|---|
| 0 | 6 | 6 | 6 | 6 |
| 5 | 6 | 6 | 6 | 6 |
| 10 | 6 | 6 | 6 | 6 |
| 15 | 6 | 6 | 6 | 6 |

| | |
|---|---|
| Total mice | 96 |

Scaffold A = A collagen scaffold.
Scaffold X = Synthes XCM ™ scaffold

After 2 and 4 weeks, muscle tissues were recovered by necropsy. Radiography was performed to determine the ossification and digital images were recorded for further analysis. All tissue samples were fixed with 10% neutral buffered formalin for 48 hours. Fresh 10% neutral buffered formalin was replaced after 48 hours and tissues were stored in this buffer until histological assessment.

### In Vivo Ectopic Ossification by Radiography

Representative images from the in vivo ectopic ossification mouse models were obtained at 2 weeks with the respective doses of rhBMP-2 delivered. There were 47 two-week specimens; 1 mortality resulted from the original 48 surgeries. The specimens are in 10% neutral buffered formalin and will be prepared in the Sakura Tissue Tek VIP E300 in formalin to ensure complete fixation. Afterwards, four samples (out of six) from each cohort, 31 samples in total, were decalcified in formic acid and endpoint tests performed to control the decalcification and prevent over-decalcification. These demineralized samples are processed as ‘soft tissue’, dehydrated in ethanol and xylene and embedded in paraffin for sectioning. Sections will be stained using hematoxylin and eosin. The remaining 16 samples were processed un-decalcified; i.e., dehydrated with ethanol and xylene and embedded in polymethyl methacrylate. These slides will be stained using von Kossa and Sanderson's rapid bone stain.

The four-week specimens, 48 in number, will be treated in the same way as stated above. The resulting slides will be analyzed to determine the degree of ossification. The histological process is estimated to take 3-4 weeks for the decalcified tissues and 10-12 weeks for the undecalcified tissues to complete.

We claim:

1. A nanostructured bioconjugate comprising:
a polymeric nanostructure formed using a controlled radical polymerization process, the polymeric nanostructure comprising:
a cationic region;
at least one degradable unit formed by incorporation of a divinyl monomeric unit, wherein the vinyl units are connected directly or indirectly by a degradable linking group; and
at least one moiety selected from the group consisting of a covalently incorporated tertiary amine moiety, a covalently incorporated quaternary ammonium moiety, and combinations of any thereof;
and
a nucleic acid at least partially encapsulated by the cationic region of the polymeric nanostructure.

2. The nanostructured bioconjugate of claim 1, wherein the nucleic acid comprises a short interfering ribonucleic acid (siRNA).

3. The nanostructured bioconjugate of claim 2, wherein the siRNA is selected to inhibit an RNA from the group consisting of a Runx2 mRNA, Osx mRNA, BMP type I receptor mRNA, BMP type II receptor mRNA, TAZ mRNA, PLZF mRNA, SMAD4 mRNA and combinations of any thereof.

4. The nanostructured bioconjugate of claim 2, wherein the siRNA has a nucleic acid sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15 and combinations of any thereof.

5. The nanostructured bioconjugate of claim 2, wherein the cationic region interacts with the siRNA by electrostatic interactions.

6. The nanostructured bioconjugate of claim 1, wherein the degradable linking group is a degradable group selected from the group consisting of a disulfide group, an ester group, an acetal group, and combinations of any thereof.

7. The nanostructured bioconjugate of claim 1, wherein the controlled radical polymerization process is an atom transfer radical polymerization (ATRP) process.

8. The nanostructured bioconjugate of claim 1, wherein the polymeric nanostructure degrades to a degradation unit comprising a primary polymer chain length defined by the molar ratio of co-monomers to initiator and is in the range of Mn <30,000.

9. The nanostructured bioconjugate of claim 1, further comprising a water soluble neutral polymeric layer comprising water soluble monomer units.

10. The nanostructured bioconjugate of claim 9, wherein the water soluble monomer units are polyethylene oxide).

11. The nanostructured bioconjugate of claim 9, wherein a peripheral functionality of the water soluble neutral polymeric layer delivers the nucleic acid to a targeted biological location.

12. The nanostructured bioconjugate of claim 1, wherein the polymeric nanostructure is a star copolymer having size in the range of about 10 to about 50 nm.

13. The nanostructured bioconjugate of claim 9, wherein the nanostructured bioconjugate comprises functionalized polymeric arms, wherein the polymeric arms comprise moieties used to target specific cells.

14. The nanostructured bioconjugate of claim 12, wherein the star copolymer comprises functionalized polymeric arms, wherein the polymeric arms comprise moieties used to target specific cells.

15. The nanostructured bioconjugate of claim 1, wherein the polymeric nanostructure is a nano-gel structure having size in the range of about 25 to about 500 nm.

16. A method of treating a clinical condition comprising:
delivering a nucleic acid to a targeted biological location using a nanostructured bioconjugate comprising:
a polymeric nanostructure formed using a controlled radical polymerization process, the polymeric nanostructure comprising:
a cationic region;
at least one degradable unit formed by incorporation of a divinyl monomeric unit, wherein the vinyl units are connected directly or indirectly by a degradable linking group; and
at least one moiety selected from the group consisting of a covalently incorporated tertiary amine moiety, a covalently incorporated quaternary ammonium moiety, and combinations of any thereof;
and
a nucleic acid at least partially encapsulated by the cationic region of the polymeric nanostructure.

17. The method of claim 16, wherein the clinical condition is selected from the group consisting of a pathological condition, an oncological condition, a genetic condition, and a vectoral condition.

18. The method of claim 16, wherein the clinical condition is heterotopic ossification.

19. The method of claim 16, wherein the nucleic acid comprises siRNA.

20. The method of claim 19, wherein the nucleic acid is an siRNA selected to inhibit an RNA from the group consisting of a Runx2 mRNA, Osx mRNA, BMP type I receptor mRNA, BMP type II receptor mRNA, TAZ mRNA, PLZF mRNA, SMAD4 mRNA and combinations of any thereof.

21. The method of claim 19, wherein the nucleic acid has a nucleic acid sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15 and combinations of any thereof.

22. The method of claim 16, further comprising co-administering a matrix forming compound, wherein the nanostructured bioconjugate and the matrix forming compound form a porous three-dimensional nano-structured network hydrogel.

23. The method of claim 22, wherein the matrix forming compound is selected from the group consisting of thiolated hyaluronic acid, thiolated collagen, non-thiolated collagen and combinations of any thereof.

24. The method of claim 22, wherein the nanostructured bioconjugate and the matrix forming compound react to form a porous three-dimensional nano-structured network hydrogel connected by a plurality of degradable cross-linking connections.

25. The method of claim 24, wherein the nano-structured network hydrogel is capable of delayed delivery of the nucleic acid by degradation of at least one of the matrix forming compound and the degradable cross-linking groups.

26. A nano-structured network hydrogel comprising:
a nanostructured bioconjugate comprising:
a polymeric nanostructure formed using a controlled radical polymerization process, the polymeric nanostructure comprising:
a cationic region;
at least one degradable unit formed by incorporation of a divinyl monomeric unit, wherein the vinyl units are connected directly or indirectly by a degradable linking group; and
at least one moiety selected from the group consisting of a covalently incorporated tertiary amine moiety, a covalently incorporated quaternary ammonium moiety, and combinations of any thereof;
and
a nucleic acid at least partially encapsulated by the cationic region of the polymeric nanostructure;
and
a matrix forming compound;
wherein the nanostructured bioconjugate and the matrix forming compound form a porous three-dimensional nano-structured network hydrogel.

27. The nano-structured network hydrogel of claim 26, wherein the nanostructured bioconjugate comprises a nano-gel structure and a nucleic acid.

28. The nano-structured network hydrogel of claim 26, wherein the nanostructured bioconjugate comprises a star copolymer and a nucleic acid.

29. The nano-structured network hydrogel of claim 26, wherein the matrix forming compound is selected from the group consisting of thiolated hyaluronic acid, thiolated collagen, non-thiolated collagen and combinations of any thereof.

30. The nano-structured network hydrogel of claim 26, wherein the nanostructured bioconjugate and the matrix forming compound react to form a porous three-dimensional nano-structured network hydrogel connected by a plurality of degradable cross-linking connections.

31. The nano-structured network hydrogel of claim 30, wherein the nano-structured network hydrogel is capable of delayed delivery of the nucleic acid by degradation of at least one of the matrix forming compound and the degradable cross-linking groups.

32. The nano-structured network hydrogel of claim 26, wherein the matrix forming compound comprises non-thiolated collagen.

33. A method of treating a clinical condition comprising:
administering to a patient having a clinical condition a nanostructured bioconjugate comprising:
a polymeric nanostructure formed using a controlled radical polymerization process, the polymeric nanostructure comprising:
a cationic region;
at least one degradable unit formed by incorporation of a divinyl monomeric unit, wherein the vinyl units are connected directly or indirectly by a degradable linking group; and
at least one moiety selected from the group consisting of a covalently incorporated tertiary amine moiety, a covalently incorporated quaternary ammonium moiety, and combinations of any thereof;
and
a nucleic acid at least partially encapsulated by the cationic region of the polymeric nanostructure;
administering to the patient a matrix forming compound; and
forming a nano-structured network hydrogel;
wherein the nano-structured network hydrogel and the nanostructured bioconjugate degrade releasing the nucleic acid to a localized targeted biological site.

34. The method of claim 33, wherein the nano-structured network hydrogel comprises a plurality of degradable cross-linking connections formed in vivo by reacting the nanostructured bioconjugate and the matrix forming compound at the localized targeted biological site.

35. The method of claim 33, wherein the matrix forming compound is selected from the group consisting of thiolated hyaluronic acid, thiolated collagen, and combinations of any thereof.

36. The method of claim 33, wherein the matrix forming compound comprises non-thiolated collagen.

37. The method of claim 33, wherein a different ratio of the nucleic acid in the nanostructured bioconjugate and matrix forming compound is used.

38. The method of claim 33, wherein the nucleic acid is siRNA capable of at least one of abrogating bone morphogenetic protein signaling and preventing heterotopic ossification.

39. The method of claim 38, wherein the siRNA inhibits mRNA expression in cells selected from the group consisting of mesenchymal cells and osteoblast lineage cells.

40. The method of claim 38, wherein the siRNA inhibits mRNA selected from the group consisting of Runx2 mRNA, Osx mRNA, BMP type I receptor mRNA, BMP type II receptor mRNA, TAZ mRNA, PLZF mRNA, SMAD4 mRNA and combinations of any thereof.


SEQUENCE LISTING (15 sequences)

SEQ ID NO: 1 - RNA, 21 nt, Artificial sequence
  Runx1 siRNA
ccgccgcuucacgccgccuuc

SEQ ID NO: 2 - RNA, 21 nt, Artificial sequence
  Runx2 siRNA
ggcugcaagcaguauuuacuu

SEQ ID NO: 3 - RNA, 21 nt, Artificial sequence
  Osx siRNA
gccauacgcugaccuuucauu

SEQ ID NO: 4 - RNA, 21 nt, Artificial sequence
  Osx siRNA
gggcacagcucgucugacuuu

SEQ ID NO: 5 - RNA, 21 nt, Artificial sequence
  Osx siRNA
caacacaccuacuccuugguu

SEQ ID NO: 6 - RNA, 21 nt, Artificial sequence
  Osx siRNA
gcaggcaucucaccaggucuu

SEQ ID NO: 7 - RNA, 25 nt, Artificial sequence
  BMP Type I receptor siRNA
gaagugcuggaugaaagccugaaua

SEQ ID NO: 8 - RNA, 21 nt, Artificial sequence
  BMP type II receptor siRNA
gggagcacguguuauggucuu

SEQ ID NO: 9 - RNA, 21 nt, Artificial sequence
  TAZ siRNA
aaucaccacauggcaagacuu

SEQ ID NO: 10 - RNA, 21 nt, Artificial sequence
  TAZ siRNA
gucuugccauguggugauuuu

SEQ ID NO: 11 - RNA, 21 nt, Artificial sequence
  TAZ siRNA
agagauacuuccuuaaucauu

SEQ ID NO: 12 - RNA, 21 nt, Artificial sequence
  TAZ siRNA
ugauuaaggaaguaucucuuu

SEQ ID NO: 13 - RNA, 21 nt, Artificial sequence
  PLZF siRNA
cagccgcaucugguuggccuu

SEQ ID NO: 14 - RNA, 21 nt, Artificial sequence
  PLZF siRNA
gaagaggaucucaaacaucuu

SEQ ID NO: 15 - RNA, 21 nt, Artificial sequence
  SMAD 4 siRNA
cugacgcaaaucaaagaccuu